(19) United States
(12) Patent Application Publication
(10) Pub. No.: US 2019/0247371 A1
(43) Pub. Date: Aug. 15, 2019

(54) METHODS FOR TREATING SMALL CELL LUNG CANCERS BY USING PHARMACEUTICAL COMPOSITIONS OR COMBINATIONS COMPRISING INDOLIZINO[6,7-B]INDOLE DERIVATIVES

(71) Applicants: Tsann-Long SU, Amawalk, NY (US); ACADEMIA SINICA, Taipei (TW)

(72) Inventors: Tsann-Long SU, Amawalk, NY (US); Te-Chang LEE, Taipei (TW)

(73) Assignees: ACADEMIA SINICA, Taipei (TW); Tsann-Long SU, Amawalk, NY (US)

(21) Appl. No.: 16/335,765

(22) PCT Filed: Sep. 21, 2017
(86) PCT No.: PCT/US2017/052675

§ 371 Date: Mar. 22, 2019

Related U.S. Application Data
(60) Provisional application No. 62/398,293, filed on Sep. 22, 2016.

(51) Int. Cl. A61K 31/437; A61K 45/06; A61P 35/00

(52) U.S. Cl. CPC A61K 31/437; A61P 35/00; A61K 45/06

(57) ABSTRACT

A method for treating small cell lung cancer (SCLC). In the method, a therapeutically effective amount of a compound of Formula I:

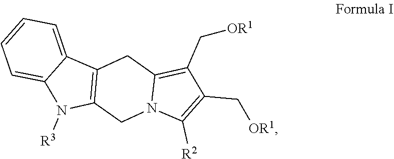

wherein R¹, R² and R³ have the definitions disclosed in the specification is administered alone or in combination with one or more anticancer agents, or surgery, radiation therapy, chemotherapy, and/or targeted therapy.

### CROSS REFERENCE

This application claims the benefit of U.S. Provisional Patent Application No. 62/398,293, filed on Sep. 22, 2016, the entire content of which is incorporated by reference.

### FIELD OF THE INVENTION

The present invention relates to a use of an indolizino[6,7-b]indole derivative in the treatment of small cell lung cancer (SCLC) in a subject in need thereof.

### BACKGROUND OF THE INVENTION

Lung cancer is identified as the leading cause of tumor-related deaths in the world (Parkin et al. 2005¹; Edwards et al. 2014²). There were 1.6 million cases of lung cancer world-wide and 224,210 new cases in the US with 159,260 deaths in 2014. The 5-year survival of patients with lung cancer was 16.8%. Lung cancer is also the most common cancer death in Taiwan. The 5 year survival is 49% for patients diagnosed at an early stage (1A), but 1% when diagnosed at stage IV. The majority (80-90%) of long cancer is due to long-term exposure to tobacco smoke (Biesalski et al. 1998³). Approximately 10-15% of cases occur in people who have never smoked. These cases are often due to a combination of genetic factors, exposure to radon gas, asbestos or other forms of air pollution, including second-hand smoke.

Lung cancers are classified according to histological type. The classification is important for determining management and predicting outcomes of the disease. Lung cancers are carcinomas—malignancies that arise from epithelial cells. Lung carcinomas are categorized by the size and appearance of the malignant cells seen by a histopathologist under a microscope. For therapeutic purposes, two broad classes are distinguished: non-small-cell lung carcinoma and small-cell lung carcinoma.

(1) Non-Small Cell Lung Cancer (NSCLC): NSCLC can be subdivided into adenocarcinoma, large cell carcinoma and squamous cell carcinoma. Adenocarcinomas are the most frequent (40% of all lung cancers) followed by squamous (30%), small-cell (13-15%), and large-cell (9-10%). Rare subtypes are giant cell carcinoma, sarcomatoid carcinoma, rhabdoid carcinoma and papillary adenocarcinoma. Bronchioalveolar carcinoma is a subtype of adenocarcinoma that occurs more frequently in female non-smokers and has a better prognosis. Numerous cell lines have been derived from these subtypes, and some lines may have features of more than one subtype e.g. adenosquamous lines. Approximately 10% of patients with NSCLC in the US and 35% in East Asia have tumor associated epidermal growth factor receptor (EGFR) mutations (Lynch et al. 2004⁴). Mutations in the K-Ras proto-oncogene are responsible for 10-30% of lung adenocarcinomas, about 4% of non-small-cell lung carcinomas involve an EML4-ALK tyrosine kinase fusion gene (Sasaki et al. 2010⁵).

Curative treatments of NSCLC include surgery, radiation therapy, chemotherapy, and targeted therapy. Among chemotherapeutic agents, cisplatin, carboplatin, mitomycin C, paclitaxel, isosfamide, doxorubicin, irinotecan, and vinorelbine are frequently used, either alone or in combination, for treatment of patients with NSCLC (Clegg et al. 2002⁶; Cataldo et al. 2011⁷). However, tumor cell resistance to chemotherapy agents (chemoresistance) remains a significant challenge in the management of human neoplasms (Chang 2011⁸).

(2) Small-Ceil Lung Carcinoma (SCLC): SCLC, the most aggressive subtype of lung cancer (Karachaliou et al. 2016⁹), is derived from neuroendocrine cells or neuroendocrine progenitors in the lung after long-term exposure to carcinogens in cigarettes (Pleasance et al. 2010¹⁰). There are two main types of small cell lung cancer; small cell carcinoma (oat cell cancer) and combined small cell carcinoma (WHO 1981¹¹). These two types include many different types of cells. The cancer cells of each type grow and spread indifferent ways. SCLC accounts for approximately 15% of bronchogenic carcinomas. At the time of diagnosis, approximately 30% of patients with SCLC will have tumors confined to the hemithorax of origin, the mediastinum, or the supraclavicular lymph nodes. These patients are designated as having limited-stage disease (LD). Patients with tumors that have spread beyond the supraclavicular areas are said to have extensive-stage disease (ED) (Murray et al. 1993¹²). Due to the improvement of analysis of pleural effusion by cytology, TNM classification has also been adopted by clinics (Shepherd et al. 2007¹³).

SCLC is a very chemosensitive tumor. A variety of chemotherapeutic agents are actively against SCLC. Platinum-based treatment and irradiation have been the standard of care since the early 1980s (Kalemkerian 2014¹⁴). An early report showed no advantage in a randomized trial of cyclophosphamide, doxorubicin, and vincristine versus cisplatin in SCLC (Fukuoka et al. 1991¹⁵). Unfortunately, monodrug-based therapy was found to have low complete response rates. Because SCLC has a greater tendency to be widely disseminated by the time of diagnosis as well as to develop early resistance to conventional treatments, a cure is difficult to achieve (Alvarado-Luna and Morales-Espinosa 2016¹⁶). The poor prognosis of SCLC is likely due to the rapid growth, early tumor spread and unavoidable drug resistance (Byers and Rudin 2015¹⁷). Only 5% of patients are alive 2 years after diagnosis. During the past decades, randomized trials have established that combination chemotherapy provides a survival benefit over monotherapy in the first-line setting.

Combination therapy by using multidrugs for the treatment of SCLC is therefore a common strategy for SCLC treatment (Murray and Turrisi 2006¹⁸). Results have been reported of three randomized clinical trials combining cisplatin with irinotecan (Camptosar), topotecan (Hycamtin), or pemetrexed (Alimta) in the first-line setting (Noda et al. 2002¹⁹; Sundstrom et al. 2002²⁰; Eckardt et al. 2006²¹; Hanna et al. 2006²²; Heigener et al. 2009²³; Lara et al. 2009²⁴). Combinations with carboplatin, gemcitabine, paclitaxel, vinorelbine, topotecan and irinotecan have also been used (Brahmer and Ettinger 1998²⁶; Azim and Ganti 2007²⁶; Horn et al. 2009²⁷). A combination of vinorelbine, ifosfamide and cisplatin (NIP) was slightly inferior to the traditional platinum-based treatment (Luo et al. 2012²⁸). Intriguingly, a recent phase 3 randomized controlled trial indicated that thoracic radiotherapy in addition to prophylactic cranial irradiation significantly increased 2-year overall survival (13% versus 3% in the control group) (Slotman et al. 2015²⁹). In general, as compared to single drug-based treatments, the overall survival of SCLC is improved by the multidrug therapy based on alkylating agents (Livingston et al. 1978³⁰; Feld et al. 1981³¹). However, almost all patients eventually relapse with refractory disease.

Several targeting therapeutic agents are currently being investigated for use in treatment of SCLC (Santarpia et al. 2016³²). These agents targeting molecular pathways in lung cancer are available, especially for the treatment of advanced disease, including Erlotinib (Tarceva), gefitinib (Irissa) and afatinib which inhibit tyrosine kinases at the EGF receptor. Denosumab is a fully human monoclonal antibody designed to inhibit RANK ligand RANKL (Abidin et al. 2010³³). In contrast to the progress made in the treatment of NSCLC, there are no accepted regimens for SCLC patients whose disease has progressed after first- and second-line treatments (Karachaliou et al. 2016⁹). Since progress on control of SCLC has been modest in the past several decades (Koinis et al. 2016³⁴, Santarpia et al. 2016³²), there is an unmet need to identify better drug and treatment strategies for the treatment of patients with SCLC.

### Bis(hydroxymethyl)indolizino[6,7-b]indoles: Potent Antitumor Agents Against NSCLC Cells

To discover new and potential agents for the treatment of lung cancer, numerous compounds have been screened. Of these compounds, bis(hydroxymethyl)indolizino[6,7-b]indoles were designed as a hybrid molecule containing biologically active β-carboline and bis(hydroxymethyl)pyrrole pharmacophores (U.S. Pat. No. 8,703,951 B2). β-Carboline derivatives exhibit antitumor activity through DNA intercalation (Guan et al. 2006³⁵) and the inhibition of topoisomerase I (topo I) and II (topo II) (Funayama et al. 1996³⁶; Deveau et al. 2001³⁷; Guan et al. 2006³⁵), cyclin-dependent kinase (CDK) (Song et al. 2004³⁸) and IkK kinase complex (IkK) (Castro et al. 2003³⁹). Bis(hydroxymethyl)pyrrole has been reported to induce DNA cross-linking (Anderson and Halat 1979⁴⁰; Anderson et al. 1980⁴¹). It is demonstrated that these indolizino[6,7-b]indole derivatives are potent anticancer agents that significantly suppress the growth of human breast carcinoma MX-1, lung adenocarcinoma A549, and colon cancer HT-29 xenograft tumor models (U.S. Pat. 8,703,951 B2). Among these hybrids, it is found that the compound [3-ethyl-6-methyl-6,11-dihydro-5H-indolizino[6,7-b]indole-1,2-diyl]dimethanol (BO-1978, FIG. 1) exhibits significant cytotoxicity against the cell growth of various NSCLC cells in vitro and potent therapeutic efficacy in nude mice bearing NSCLC in xenograft and orthotopic models (Chen et al. 2016⁴²). In brief, it is reported that BO-1978 significantly suppresses the growth of various NSCLC cell lines with or without mutations in EGFR. Mechanistically, it is demonstrated that BO-1978 exhibits multiple modes of action, including inhibition of topo I/II and induction of DNA cross-linking. Treatment of NSCLC cells with BO-1978 causes DNA damage, disturbs cell cycle progression, and triggers apoptotic cell death. Furthermore, BO-1978 significantly suppresses the growth of EGFR wild-type and mutant NSCLC tumors in xenograft tumor and orthotopic lung tumor models with negligible body weight loss. These results imply that BO-1978 and its derivatives are potential chemotherapeutic agents against NSCLC with wild-type or mutant EGFR. Although tyrosine kinase inhibitors (TKIs) are promising drugs against EGFR mutant NSCLC (Zarogoulidis et al. 2013⁴³), the generation of resistance to TKIs is a major reason that treatments fail. Intriguingly, BO-1978 also effectively kills the cells that acquire gefitinib resistance, PC9/gef B4 cells. The combination of BO-1978 with gefitinib further suppresses EGFR mutant NSCLC cell growth in xenograft tumor and orthotopic lung tumor models. Preclinical toxicity studies show that BO-1978 administration does not cause apparent toxicity in mice. In addition, it is found that BO-1978 shows no cross-resistance to multiple drug-resistant and cisplatin-resistant cells. Based on its significant therapeutic efficacy and low drug toxicity, BO-1978 is a potential therapeutic agent for treatment of NSCLC.

It is demonstrated that BO-1978 significantly suppresses the growth of EGFR wild-type and mutant NSCLC tumors in xenograft tumor and orthotopic lung tumor models with negligible body weight loss (Chen et al. 2016⁴²). The combination of BO-1978 with gefitinib further suppresses EGFR mutant NSCLC cell growth in xenograft tumor and orthotopic lung tumor models. In addition, a previous study (Chen et al. 2016⁴²) has shown that BO-1978 could overcome the multidrug resistance.

Decreased incidence of SCLC has been observed in the United Stales over the last 30 years (12.95% of all newly diagnosed lung cancers) (Govindan et al. 2006⁴⁵). In contrast, the number of abstracts for NSCLC has skyrocketed. The slow pace of SCLC investigation is unfortunate and puzzling because the proportion of estimated deaths from this disease is approximately 4% of all cancer mortality (Jemal et al. 2005⁴⁶). That may be attributed to patients with SCLC tending to develop distant metastases, and thus, the localized forms of treatment, such as surgical resection or radiation therapy, rarely produce long-term survival (Prasad et al. 1989⁴⁷).

Consequently, more clinical and basic research and discovery of new drugs for the treatment of SCLC is required.

### SUMMARY OF THE INVENTION

One aspect of the present invention is to provide a method for the treatment of SCLC in a subject in need thereof comprising administering to the subject a therapeutically effective amount of a compound of Formula I.

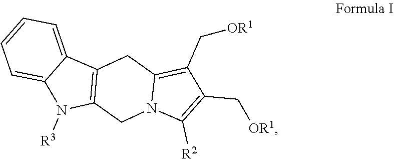

or an enantiomer, diastereomer, racemate, pharmaceutically acceptable salt, solvate or prodrug thereof.
wherein R¹, R² and R³ have the definitions described below.

Another aspect the present invention is to provide a method for the treatment of SCLC in a subject in need thereof comprising administering to the subject a therapeutically effective amount of a compound of Formula I or an enantiomer, diastereomer, racemate, pharmaceutically acceptable salt, solvate or prodrug thereof in combination with a second anticancer agent, a surgical therapy, a radiation therapy, a chemotherapy, a targeted therapy, or combination thereof.

Another aspect of the invention is to provide a use of the compound of Formula I, or an enantiomer, diastereomer, racemate, pharmaceutically acceptable salt, solvate or prodrug thereof in the manufacture of a medicament for the treatment of SCLC, wherein the medicament can be administered to a subject alone or in combination with a second anticancer agent, a surgical therapy, a radiation therapy, a chemotherapy, a targeted therapy, or combination thereof.

Another aspect of the invention is to provide a compound of Formula I, or an enantiomer, diastereomer, racemate, pharmaceutically acceptable salt, solvate or prodrug thereof for the treatment of SCLC.

Another aspect of the invention is to provide a pharmaceutical composition for treating SCLC comprising the compound of Formula I, or an enantiomer, diastereomer, racemate, pharmaceutically acceptable salt, solvate or prodrug thereof and one or more pharmaceutically acceptable excipients.

Another aspect of the invention is to provide a combination comprising the compound of Formula I, or an enantiomer, diastereomer, racemate, pharmaceutically acceptable salt, solvate or prodrug thereof and a second anticancer agent. These and other aspects of the present invention will become apparent from the following detailed description.

### BRIEF DESCRIPTION OF THE DRAWINGS

FIG. 1 shows the chemical structure of BO-1978.

FIG. 2 shows the therapeutic effects of BO-1978 against small cell lung cancer H526 cells. H526 cells (1×10⁷) were subcutaneously implanted into nude mice. When tumor size reached 100-200 mm³, BO-1978, etoposide, irinotecan, and cisplatin were i.v. administered as indicated. Tumor size and body weight were taken at the time indicated. A, average tumor size changes; B, average body weight changes; and C, Kaplan-Meyer survival curves.

FIG. 3 shows the therapeutic effects of BO-1978 against small cell lung cancer H211 cells. H211 (3×10⁶) were subcutaneously implanted into nude mice. When rumor size reached 100-200 mm³, BO-1978, etoposide, irinotecan, and cisplatin were i.v. administered as indicated. Tumor size and body weight were taken at the time indicated. A, tumor size; and B, body weight.

FIG. 4 shows cytotoxicity of combined treatment of BO-1978 with cisplatin, etoposide, or irinotecan to H526 cells H526 cells were seeded and treated with various concentrations of BO-1978, or therapeutic agents either alone or in combination for 72 h. Cell survival was analyzed using alamarBlue assay. CI indicates combination index A, BO-1978 and cisplatin; B, BO-1978 and etoposide; and C, BO-1978 and irinotecan.

FIG. 5 shows the cytotoxicity of combined treatment of BO-1978 with cisplatin, etoposide, or irinotecan to H211 cells. H211 cells were seeded and treated with various concentrations of BO-1978, or therapeutic agents either alone or in combination for 72 h. Cell survival was analyzed using alamarBlue assay. CI indicates combination index. A, BO-1978 and cisplatin; B, BO-1978 and etoposide; and C, BO-1978 and irinotecan.

FIG. 6 shows the therapeutic effects of BO-1978 and irinotecan against small cell lung cancer H526 cells. H526 cells (1×10⁷) were subcutaneously implanted into nude mice. When tumor size reached 100-200 mm³, BO-1978 and irinotecan, either alone or in combination were i.v. administered as indicated. Tumor size and body weight were taken at the time indicated A, tumor size; and B, body weight.

FIG. 7 shows the therapeutic effects of BO-1978 and irinotecan against small cell lung cancer H211 cells. H211 cells (3×10⁶) were subcutaneously implanted into nude mice. When tumor size reached 100-200 mm³, BO-1978 and irinotecan, cither alone or in combination were i.v. administered as indicated. Tumor size and body weight were taken at the time indicated. A, tumor size; and B, body weight.

FIG. 8 shows the synergistic effect of the combination of BO-1978 with a PARP inhibitor (BMN-673 or HY-10130) in the suppression of the growth of SCLC H211 cells. H211 cells were seeded and treated with various concentrations of BO-1978, BMN-673, or HY-10130 either alone or in combination for 72 h. Cell survival was analyzed using PrestoBlue assay. CI indicates combination index. A, BO-1978 and BMN-673; B, BO-1978 and HY-10130.

FIG. 9 shows the cell cycle progression interference effect caused by BO-1978 and BMN-673, either alone or in combination, in SCLC H211 cells. H211 cells were treated with various concentrations of (A) BO-1978, (B) BMN-673, or (C) BO-1978 plus BMN-673 at 24, 48, and 72 h. At the end of treatment, the cells were harvested by trypsinization, fixed in ice-cold ethanol, stained with propidium iodide, and subjected to cell cycle analysis with a flow cytometer.

FIG. 10 shows the synergistic effect of the combination of BO-1978 with a PI3K inhibitor (LY-294002) in the suppression of the growth of SCLC H211 and H526 cells. (A) H211 and (B) H526 cells were seeded and treated with various concentrations of BO-1978 or LY-294002, either alone or in combination for 72 h. Cell survival was analyzed using PrestoBlue assay. CI indicates combination index.

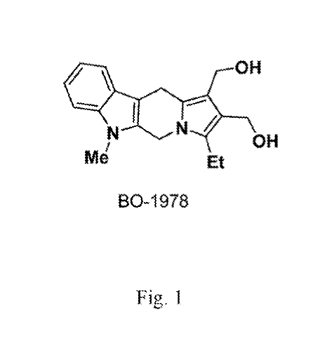

### DETAILED DESCRIPTION OF THE INVENTION

The present invention can be understood more readily by reference to the following detailed description of various embodiments of the invention, the examples, and the tables with their relevant descriptions. Unless defined otherwise, all technical and scientific terms used herein have the same meaning as commonly understood by one of ordinary skill in the art to which this invention belongs. It will be further understood that terms such as those defined in commonly used dictionaries should be interpreted consistently with their meaning in the context of the relevant art and will not be interpreted in an idealized or overly formal sense unless expressly so defined herein. It is also to be understood that the terminology used herein is for the purpose of describing particular embodiments only and is not intended to be limiting. Although any methods and materials similar or equivalent to those described herein can be used in the practice or testing of the invention, the preferred methods and materials are now described. All publications mentioned herein are incorporated herein by reference.

The definitions set forth in this section are intended to clarify terms used throughout this application. In this section, the definitions apply to the compounds of Formula I unless otherwise stated. The term “herein” means the entire application.

It must be noted that, as used herein, the singular forms “a,” “an” and “the” include plural referents unless the context clearly dictates otherwise. Thus, unless otherwise required by context, singular terms shall include the plural and plural terms shall include the singular.

The word “or” in reference to a list of two or more items, covers all of the following interpretations of the word: any of the items in the list, all of the items in the list, and any combination of the items in the list.

Often, ranges are expressed herein as from “about” one particular value and/or to “about” another particular value. When such a range is expressed, an embodiment includes the range from the one particular value and/or to the other particular value. Similarly, when values are expressed as approximations, by use of the word “about,” it will be understood that the particular value forms another embodiment. It will be further understood that the endpoints of each of the ranges are significant both in relation to and independently of the other endpoint. As used herein, the term “about” refers to ±20%, preferably ±10%, and even more preferable ±5%.

In this application, the word “comprise,” or variations such as “comprises” or “comprising,” indicate the inclusion of any recited integer or group of integers but not the exclusion of any other integer or group of integers in the specified method, structure, or composition.

The present invention provides a method for treating SCLC in a subject in need thereof comprising administering to the subject a therapeutically effective amount of a compound of Formula I:

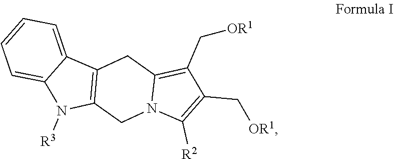

wherein

R¹ is selected from hydrogen or —C(═O)NHR, wherein R is unsubstituted or substituted alkyl, unsubstituted or substituted alkenyl, unsubstituted or substituted alkynyl, unsubstituted or substituted cycloalkyl, unsubstituted or substituted aryl, unsubstituted or substituted heteroaryl, and unsubstituted or substituted benzyl;

R² is selected from the group consisting of hydrogen, unsubstituted or substituted alkyl, unsubstituted or substituted alkenyl, unsubstituted or substituted alkynyl, unsubstituted or substituted cycloalkyl, unsubstituted or substituted aryl, unsubstituted or substituted heteroaryl, and unsubstituted or substituted benzyl; and

R³ is selected from the group consisting of hydrogen, unsubstituted or substituted alkyl, unsubstituted or substituted alkenyl, unsubstituted or substituted alkynyl, unsubstituted or substituted cycloalkyl, unsubstituted or substituted benzyl, an acyl (R^{a}CO), a methansulfonyl (Me₂SO₂) and a toluenesulfonyl (MeC₆H₄SO₂); wherein R^{a} is unsubstituted or substituted alkyl, unsubstituted or substituted alkenyl, unsubstituted or substituted alkynyl, unsubstituted or substituted cycloalkyl, unsubstituted or substituted aryl, unsubstituted or substituted heteroaryl, and unsubstituted or substituted benzyl,

or an enantiomer, diastereomer, racemate, pharmaceutically acceptable salt, solvate or prodrug thereof.

As used herein, the phrase “unsubstituted or substituted” means that substitution is optional. In the event a substitution is desired, then such substitution means that any number of hydrogen atoms on the designated atom are replaced with a selection from the indicated group, provided that the normal valence of the designated atom is not exceeded, and that the substitution results in a stable compound. For example, when a substituent is a keto group (i.e., ═O), then two hydrogens on the atom are replaced. Examples of substituents for a “substituted” group can include, for example, halogen, hydroxy, amino, acetylamino, carboxy, cyano, haloalkyl, alkyl, alkenyl, alkynyl, cycloalkyl, alkoxy, haloalkyl, alkylamino, aminoalkyl, dialkylamino, hydroxylalkyl, alkoxyalkyl, hydroxyalkoxy, alkoxyalkoxy, aminoalkoxy, alkylaminoalkoxy, alkylaminoalkyl, heterocyclic, aryl, heteroaryl and the like.

The term “hydrocarbon” used herein refers to any structure comprising only carbon and hydrogen atoms and up to 12 carbon atoms.

As used herein, the term “alkyl” used herein refers to a monovalent, saturated, straight or branched hydrocarbon radical containing 1 to 12 carbon atoms. Preferably, the alkyl is a C₁-C₆ alkyl group. More preferably, the alkyl is a C₁-C₅ alkyl group. Examples of a C₁-C₆ alkyl group include, but are not limited to, methyl, ethyl, n-propyl, isopropyl, n-butyl, isobutyl, sec-butyl, t-butyl, pentyl (including all isomeric forms), hexyl (including all isomeric forms) and the like.

The term “alkenyl” used herein refers to an unsaturated, straight or branched chain hydrocarbon radical having at least one carbon-carbon double bond and comprising 2 to 12 carbon atoms. Preferably, the alkenyl is a C₂-C₆ alkenyl group. More preferably, the alkenyl is a C₂-C₅ alkenyl group. Examples of an alkenyl group include, but are not limited to, ethenyl, propenyl, butenyl, 1,4-butadienyl and the like.

The term “alkynyl” used herein refers to an unsaturated, straight or branched chain hydrocarbon radical having at least one carbon-carbon triple bond and comprising 2 to 12 carbon atoms. Preferably, the alkynyl is a C₂-C₆ alkynyl group. More preferably, the alkynyl is a C₂-C₅ alkynyl group. Examples of an alkynyl group include, but are not limited to, ethynyl, propynyl, butynyl and the like.

The term “cycloalkyl” used herein refers to a saturated, monovalent hydrocarbon radical having cyclic configurations, including monocyclic, bicyclic, tricyclic, and higher multicyclic alkyl radicals (and, when multicyclic, including fused and bridged bicyclic and spirocyclic moieties) wherein each cyclic moiety has from 3 to 12 carbon atoms. Preferably, the cycloalkyl has from 3 to 8 carbon atoms. More preferably, the cycloalkyl has from 3 to 6 carbon atoms. When cycloalkyl contains more than one ring, the rings may be fused or unfused and include bicyclo radicals. Fused rings generally refer to at least two rings sharing two atoms therebetween. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as adamantyl and the like.

The term “aryl” used herein refers to a hydrocarbon radical having one or more polyunsaturated carbon rings and a conjugated pi election system and comprising from 6 to 14 carbon atoms, wherein the radical is located on a carbon of the aromatic ring. In some embodiments, the aryl group contains from 6 to 12 carbon atoms, preferably 6 to 10 carbon atoms, in the ring portions of the groups. Exemplary aryl includes, but is not limited to, phenyl, biphenyl, naphthyl, indenyl and the like.

The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quarternized. A heteroaryl group can be attached to the remainder of the molecule through a carbon or heteroatom. Non-limiting examples of heteroaryl groups include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

In a preferred embodiment, the substituents for each of the aryl, heteroaryl and benzyl ring systems are varied and are selected from, for example, C₁-C₆alkyl, OR^{a}, halo, cyano, nitro, NH₂, NHR^{b}, N(R^{b})₂, a C₃-C₆cyclic alkylamino group, a methylenedioxy and ethylenedioxy group; wherein R^{a} is hydrogen or C₁-C₁₀alkyl, and R^{b} is hydrogen or C₁-C₁₀alkyl.

As used herein, the term “halogen” includes fluorine, chlorine, bromine and iodine. “Halo,” used as a prefix of a group, means one or more hydrogens on the group are replaced with one or more halogen.

The term “amino” refers to the —NH₂ group. “Amino,” used as a prefix or suffix of a group, means one or more hydrogens on the group are replaced with one or more amino groups.

The term “alkoxy,” used alone or as a suffix or prefix, refers to radicals of the general formula —O(alkyl), wherein alkyl is defined above. Exemplary alkoxy includes, but is not limited to, methoxy, ethoxy, n-propoxy, isopropoxy, n-butoxy, iso-butoxy, sec-butoxy, tert-butoxy, and the like.

An “alkoxyalkyl” group is represented by —(alkyl)-O-(alkyl), wherein alkyl is defined above.

The terms “heterocyclic” and “heterocyclo,” by themselves or in combination with other terms, represent, unless otherwise stated, cyclic versions of “heteroalkyl.” The term “heteroalkyl,” by itself or in combination with mother term, means, unless otherwise stated, a stable straight or branched chain, or cyclic hydrocarbon radical, or combinations thereof, consisting of at least one carbon atoms and at least one heteroatom selected from the group consisting of N, O, and S. Examples of “heterocyclic” and “heterocyclo” include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “hydroxyalkyl” refers to an alkyl group as described above substituted with one or more hydroxy groups.

The term “hydroxyalkoxy” refers to an alkoxy group as described above substituted with one or more hydroxy groups.

Compounds of Formula I can exist as pharmaceutically acceptable salts. As used herein, “pharmaceutical acceptable salts” refer to derivatives of the disclosed compounds wherein the parent compound is modified by making pharmaceutically acceptable acid or base salts thereof. Examples of pharmaceutically acceptable salts include, but are not limited to, mineral or organic acid salts of basic residues such us amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Nonlimiting examples of salts of compounds of the invention include, but are not limited to, hydrochloride, hydrobromide, hydroiodide, sulfate, bisulfate, 2-hydroxyethanesulfonate, phosphate, hydrogen phosphate, acetate, adipate, alginate, aspartate, benzoate, butyrate, camphorate, camphorsulfonate, citrate, digluconate, glycerolphosphate, hemisulfate, heptanoate, hexanoate, formate, succinate, malonate, fumarate, maleate, methanesulfonate, mesitylenesulfonate, naphthylenesulfonate, nicotinate, oxalate, pamoate, pectinate, persulfate, 3-phenylpropionate, picrate, pivalate, propionate, trichloroacetate, trifluoroacetate, glutamate, bicarbonate, undecanoate, lactate, citrate, tartrate, gluconate, benzene sulphonate, and p-toluenesulphonate salts.

Compounds of Formula I can exist as solvates. As used herein and unless otherwise indicated, the term “solvate” means a compound of Formula I, or a pharmaceutically acceptable salt thereof, that further includes a stoichiometric or non-stoichiometric amount of a solvent bound by non-covalent intermolecular forces. If the solvent is water, the solvate may be conveniently referred to as a “hydrate,” for example, a hemi-hydrate, a mono-hydrate, a sesqui-hydrate, a di-hydrate, a tri-hydrate, etc.

As used herein, “prodrugs” are intended to include any covalently bonded carriers that release the active parent drug according to Formula I through in vivo physiological action, such as hydrolysis, metabolism and the like, when such prodrug is administered to a subject. The suitability and techniques involved in making and using prodrugs are well known to a person of ordinary skill in the art. Prodrugs of compounds of Formula I (parent compounds) can be prepared by modifying functional groups present in the compounds in such a way that the modifications are cleaved, either in routine manipulation or in vivo, to the parent compounds. “Prodrugs” include compounds of Formula I wherein a hydroxy, amino, or sulfhydryl group is bonded to any group that, when the prodrugs are administered to a subject, cleaves to form a free hydroxyl, free amino, or free sulfhydryl group, respectively. Examples of prodrugs include, but are not limited to, derivatives and metabolites of compounds of Formula I that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds of Formula I with carboxyl functional groups are the lower alkyl (e.g., C₁-C₆) esters of the carboxylic acid. The carboxylate esters are conveniently formed by esterifying any of the carboxylic acid moieties present on the molecule.

As used herein, the term “enantiomers” refers to a pair of stereoisomers that are non-superimposable mirror images of each other. A 1:1 mixture of a pair of enantiomers is a racemic mixture. The term “enantiomers” is used to designate a racemic mixture where appropriate. “Diastereoisomers” are stereoisomers that have at least two asymmetric atoms, but which are not mirror images of each other. The absolute stereochemistry may be specified according to the Cahn-Ingold-Prelog R-S system. When a compound is a pure enantiomer, the stereochemistry at each chiral carbon may be specified by either R or S. Resolved compounds can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomers forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons chiral reagents, or resolved using conventional techniques. If the compound contains a double bond, the substituent may be E or Z configuration. If the compound contains a di-substituted cycloalkyl, the cycloalkyl substituent may have a cis- or trans-configuration.

In one preferred embodiment, R¹ is hydrogen.

In another preferred embodiment, R² is ethyl.

In another preferred embodiment, R³ is methyl.

In a more preferred embodiment, R¹ is hydrogen, R² is ethyl and R³ is methyl.

Examples of the compounds of Formula I may be:

(3-(phenyl)-6-methyl-6,11-dihydro-5H-indolizino[6,7-b]indole-1,2-diyl)dimethanol;
(3-(4-Fluorophenyl)-6-methyl-6,11-dihydro-5H-indolizino[6,7-b]indole-1,2-diyl)dimethanol;
(3-(4-Chlorophenyl)-6-methyl-6,11-dihydro-5H-indolizino[6,7-b]indole-1,2-diyl)dimethanol;
(3-(3,4-Difluorophenyl)-6-methyl-6,11-dihydro-5H-indolizino[6,7-b]indole-1,2-diyl)dimethanol; [6-Methyl-3-phenyl-6,11-dihydro-5H-indolizino[6,7-b]indole-1,2-diyl]bis(methylene) bis(ethylcarbamate);
[3-(4-Fluorophenyl)-6-methyl-6,11-dihydro-5H-indolizino[6,7-b]indole-1,2-diyl]bis(methylene) bis(ethylcarbamate);
[3-(4-Chlorophenyl)-6-methyl-6,11-dihydro-5H-indolizino[6,7-b]indole-1,2-diyl]bis(methylene) bis(ethylcarbamate);
[3-(4-Difluorophenyl)-6-methyl-6,11-dihydro-5H-indolizino[6,7-b]indole-1,2-diyl]bis(methylen) bis(ethylcarbamate); or
[3-ethyl-6-methyl-6,11-dihydro-5H-indolizino[6,7-b]indole-1,2-diyl]dimethanol.

### Pharmaceutical Compositions, Combinations, Use and Methods

The compounds of Formula I can be therapeutically administered as the neat chemical, but it may be useful to administer the compounds as a pharmaceutical composition or formulation. Thus, the present invention provides a pharmaceutical composition comprising a therapeutically effective amount of a compound of Formula I, or enantiomers, diastereomers, racemates, pharmaceutically acceptable salts, or prodrugs thereof, and one or more pharmaceutically acceptable excipients.

An “excipient” generally refers to a substance, often an inert substance, added to a pharmacological composition or otherwise used as a vehicle to further facilitate administration of a compound. Examples of excipients include, but are not limited to, inert diluents, disintegrating agents, binding agents, lubricating agents, sweetening agents, flavoring agents, coloring agents, preservatives, effervescent mixtures, and adsorbents. Suitable inert diluents include, but are not limited to, sodium and calcium carbonate, sodium and calcium phosphate, lactose, and the like. Suitable disintegrating agents include, but are not limited to, starches, such as corn starch, cross-linked polyvinyl pyrrolidone, agar, alginic acid, or a salt thereof, such as sodium alginate, and the like. Binding agents may include, but are not limited to, magnesium aluminum silicate, starches such as corn, wheat or rice starch, gelatin, methylcellulose, sodium carboxymethylcellulose, polyvinylpyrrolidone, and the like. A lubricating agent, if present, will generally be magnesium stearate and calcium stearate, stearic acid, talc, or hydrogenated vegetable oils.

The pharmaceutical compositions also may comprise suitable solid or gel phase carriers. Examples of such carriers include, but are not limited to, calcium carbonate, calcium phosphate, various sugars, starches, cellulose derivatives, gelatin, and polymers such as polyethylene glycols.

The compounds or pharmaceutical compositions can be administered in a variety of dosage forms including, but not limned to, a solid dosage form or a liquid dosage form, an oral dosage form, a parenteral dosage form, an intranasal dosage form, a suppository, a lozenge, a troche, a controlled release dosage form, a pulsed release dosage form, an immediate release dosage form, an intravenous solution, a suspension or combinations thereof. The compounds or composition can be administered, for example, by oral or parenteral routes, including intravenous, intramuscular, intraperitoneal, subcutaneous, transdermal, airway (aerosol), rectal, vaginal and topical (including buccal and sublingual) administration. The compound or pharmaceutical composition can be administered orally or rectally through appropriate formulation with carriers and/or excipients to form tablets, pills, capsules, liquids, gels, syrups, slurries, suspensions and the like. The compound or pharmaceutical composition can be administered by inhaler to the respiratory tract for local or systemic treatment of cancers.

As used herein, “therapeutically effective amount” means an amount sufficient to treat a subject afflicted with a disease or to alleviate a symptom or a complication associated with the disease. The “therapeutically effective amount” of the compound, or an enantiomer, diastereomer, racemate, pharmaceutically acceptable salt, solvate or prodrug thereof of the compound of Formula I will depend upon a number of factors, e.g., the age and weight of the subject, the precise condition requiring treatment and its severity, the nature of the formulation, and the route of administration, and will ultimately be at the discretion of the attendant physician or veterinarian.

As used herein, “treatment” and “treating” are used interchangeably. These terms refer to an approach for obtaining beneficial or desired results including, but not limited to, therapeutic benefit and/or a prophylactic benefit. Therapeutic benefit pertains to eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. “Treatment” can also mean prolonging survival as compared to expected survival if not receiving treatment. Those in need of treatment include those already with the condition or disorder as well as those prone to have the condition or disorder or those in which the condition or disorder is to be prevented.

A “subject” to be treated by the method of the present invention means either a human or non-human animal, such as primates, mammals, and vertebrates.

In this application, “small cell lung cancer” or “SCLC” can be categorized into several groups, for instance, “refractory SCLC” is that which fails to respond to first-line treatment e.g., cisplatin and carboplatin, or responds and then progresses within 90 days; “early-relapsing” SCLC initially responds to first line therapy and then progresses within 45 days; and “non-refractory SCLC” is that which initially responds to first line therapy, and then progresses during the 91-180 day period.

In another embodiment of the present invention, the compound of Formula I or an enantiomer, diastereomer, racemate, pharmaceutically acceptable salt, solvate or prodrug thereof is administered in combination with a known anti-cancer therapy. The phrase “in combination with” means that the compound of Formula I may be administered shortly before, shortly after, concurrently, or any combination of before, after, or concurrently, with other anti-cancer therapeutics.

Therefore, a combination which comprises the compound of Formula I or an enantiomer, diastereomer, racemate, pharmaceutically acceptable salt, solvate or prodrug thereof, and a second anticancer agent is also included in the present invention. According to the present invention, the compound of Formula I and the second anticancer agent comprised in the combination may be administered simultaneously either as a single composition or as two separate compositions or sequentially as two separate compositions. Thus, the compound of Formula I and the second anticancer agent may be administered simultaneously, separately or sequentially. According to the invention, the compound of Formula I is administered prior to, simultaneously with, or after one or more of the other anticancer agents.

The “second anticancer agent” can be, but is not limited to, anti-microtubule agents (such as diterpenoids and vinca alkaloids like vinorelbine); platinum coordination complexes; alkylating agents (such as nitrogen mustards like ifosphamide, oxazaphosphorines, alkylsulfonates, nitrosoureas (including 2-chloroethyl-3-sarcosinamide-1-nitrosourea (SarCNU)), busulfan, chlorambucil, cyclophosphamide, iphosphamide, melphalan, streptozocin, thiotepa, uracil nitrogen mustard, triethylenemelamine, temozolomide, and triazenes); antibiotic agents or plant alkaloids (such as cryptophycins, daunorubicin, doxorubicin, idarubicin, irinotecan, L-asparaginase, mitomycin-C, mitramycin, navelbine, paclitaxel, docetaxel, topotecan, vinblastine, vincristine, teniposide (VM-26), and etoposide (VP-16), anthracyclins, actinomycins (such as actinomycin-D) and bleomycins); topoisomerase II inhibitors (such as epipodophyllotoxins); hormones or steroids (such as 5α-reductase inhibitor, aminoglutethimide, anastrozole, bicalutamide, chlorotrianisene, diethylstilbestrol (DES), dromostanolone, estramustine, ethinyl estradiol, flutamide, fluoxymesterone, goserelin, hydroxy progesterone, letrozole, leuprolide, medroxyprogesterone acetate, megestrol acetate, methyl prednisolone, methyltestosterone, mitotane, nilutamide, prednisolone, arzoxifene (SERM-3), tamoxifen, testolactone, testosterone, trimicnolone, and zoladex); synthetics (such as all-trans retinoic acid, carmustine (BCNU), carboplatin (CBDCA), lomustine (CCNU), cis-diaminedichloroplatinum (cisplatin), dacarbazine, gliadel, hexamethylmelamine, hydroxyurea, levamisole, mitoxantrone, o,p′-dichlorodiphenyldichloroethane (o,p′-DDD) (also known as lysodren or mitotane), oxaliplatin, porfimer sodium, procarbazine, and imatinib mesylate (Gleevec®)); antimetabolites (such as chlorodeoxyadenosine, cytosine arabinoside, 2′-deoxycoformycin, fludarabine phosphate, 5-fluorouracil (5-FU), 5-fluoro-2′-deoxyuridine (5-FUdR), gemcitabine, camptothecin, 6-mercaptopurine, methotrexate, 4-methylthioamphetamine (4-MTA), thioguanine, pemetrexed, purine and pyrimidine analogues and anti-folate compounds); biologies (such as alpha interferon, BCG (Bacillus Calmette-Guerin), granulocyte colony stimulating factor (G-CSF), granulocyte-macrophage colony-stimulating factor (GM-CSF), interleukin-2, and herceptin); topoisomerase 1 inhibitors (such as camptothecins; hormones and hormonal analogues); signal transduction pathway inhibitors (such as tyrosine receptor inhibitors like erlotinib; EGFR inhibitors like gefitinib and afatinib; TNFR inhibitors like denosumab); non-receptor tyrosine kinase angiogenesis inhibitors; immunotherapeutic agents; proapoptotic agents, epigenetic or transcriptional modulators (such as histone deacetylase inhibitors); DNA replication or transcription inhibitors (such as picoplatin); DNA damage response (DDR) inhibitors (such as Poly(ADP-ribose) polymerase (PARP) inhibitors (e.g., Talazoparib ((8S,9R)-5-Fluoro-8-(4-fluorophenyl)-9-(1-methyl-1H-1,2,4-triazol-5-yl)-2,7,8,9-tetrahydro-3H-pyrido[4,3,2-de]phthalazin-3-one), Veliparib (HY-10130; 2-((R)-2-Methylpyrrolidin-2-yl)-1H-benzimidazole-4-carboxamide), Olaparib (4-[[3-[4-(cyclopropanecarbonyl)piperazine-1-carbonyl]-4-fluorophenyl]methyl]-2H-phthalazin-1-one, Niraparib 2-[4-[(3S)-piperidin-3-yl]phenyl]indazole-7-carboxamide), and Rucaparib (8-Fluoro-2-{4-[(methylamino)methyl]phenyl}-1,3,4,5-tetrahydro-6H-azepino[5,4,3-cd]indol-6-one)) or PI3K/AKT pathway inhibitors (e.g., LY294002 (2-Morpholin-4-yl-8-phenylchromen-4-one), buparlisib (5-[2,6-bis(morpholin-4-yl)pyrimidin-4-yl]-4-(trifluoromethyl)pyridin-2-amine), and alpelisib ((2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide)); and cell cycle signaling inhibitors. In a preferred embodiment, the second anticancer agent is irinotecan, etoposide, cisplatin, picoplatin, cyclophosphamide, doxorubicin, vincristine, topotecan, pemetrexed, carboplatin, gemcitabine, paclitaxel, vinorelbine, ifosphamide, erlotinib, gefitinib, afatinib, denosumab, Talazoparib, Veliparib, or LY294002. In a more preferred embodiment, the second anticancer agent is irinotecan, etoposide, cisplatin, Talazoparib, Veliparib, or LY294002.

In a further embodiment of the present invention, the compound of Formula I, or an enantiomer, diastereomer, racemate, pharmaceutically acceptable salt, solvate or prodrug thereof is administered in combination with surgery, radiation therapy, chemotherapy, and/or targeted therapy.

Without further elaboration, it is believed that one skilled in the art can utilize the present invention to its fullest extent on the basis of the preceding description. The following examples are, therefore, to be construed as merely illustrative and not a limitation of the scope of the present invention in any way.

### EXAMPLES

### Example 1 BO-1978 Exhibits Potent Cytotoxicity Against SCLC in Vitro

The inhibitory effect of BO-1978 on the cell growth of SCLC cell lines in vitro was examined. The cytotoxicity was assayed using alamarBlue® reagent (AbD Serotec) as previously described in U.S. Pat. No. 8,703,951 B2. In brief, H82, H211 and H526 cells were seeded to a 96 well plate with 5000, 1000 and 3000 cells per well, respectively, and incubated for 24 h. The growing cells were treated with BO-1978, cisplatin, etoposide or irinotecan at serial-diluted concentrations for 72 h at 37° C. An aliquot of alamarBlue® reagent (AbD Serotec) was added and the cultures were incubated for 5 h. The fluorescence at excitation 530 nm and emission 590 nm was read with a plate reader. The proliferation rate was calculated according to the manufacturer's instruction. The values of IC₅₀ (50% inhibition concentration) for each compound were determined from dose-effect relationship using the CompuSyn software (Chou 2010⁴⁴). The results are summarized in Table 1 below. It is revealed that BO-1978 has competitive cytotoxicity with other therapeutic agents (e.g., Cisplatin, Etoposide, and Irinotecan) currently used for the treatment of SCLC.

TABLE 1
Comparative IC₅₀ (μM) of BO-1978 with other therapeutic agents in
SCLC^{a}.

| | Compound | | |
|---|---|---|---|
| | H82 | H211 | H526 |
| BO-1978 | 0.35 ± 0.28 | 0.148 ± 0.001 | 0.076 ± 0.026 |
| Cisplatin | 0.34 ± 0.08 | 0.57 ± 0.29 | 0.66 ± 0.25 |
| Etoposide | 0.146 ± 0.002 | 0.07 ± 0.03 | 0.05 ± 0.02 |
| Irinotecan | 1.20 ± 0.07 | 0.03 ± 0.01 | 1.18 ± 0.57 |

^{a}IC₅₀, the concentration or drug required to inhibit cell growth by 50% (mean ± S.D. of 3 independent experiments).

### Example 2 BO-1978 Exhibits Potent Cytotoxicity Against SCLC in Xenograft Models

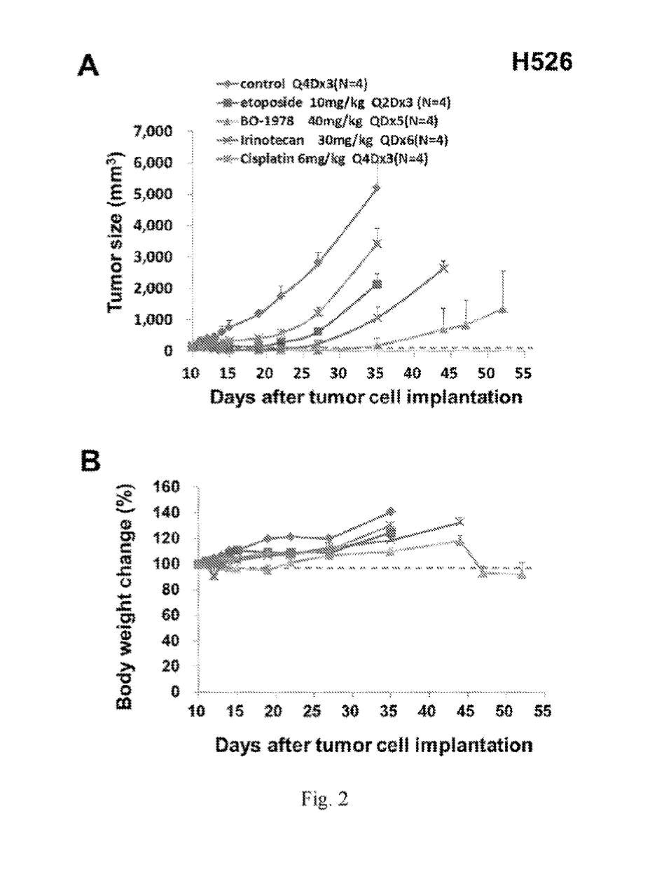

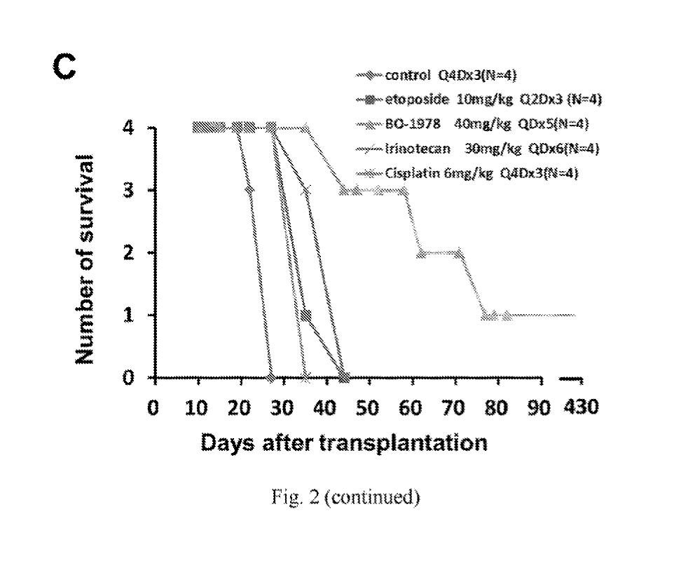

The therapeutic effects of BO-1978 against SCLC H526 cells were further examined using tumor xenograft model. The animals used in this study were treated exactly according to the guidelines of the Academia Sinica Institutional Animal Care and Utilization Committee. Male athymic nude mice in 5 weeks of age were obtained from the National Laboratory Animal Center (Taipei, Taiwan) and housed for 1 week before performing experiments H526 cells (1×10⁷) were subcutaneously implanted into nude mice (five groups, each 4 mice). When tumor size reached 100-200 mm³, BO-1978, 40 mg/kg, once per day for 5 times (QD×5) was administered via intravenous injection (iv inj.). Etoposide (10 mg/kg Q2D×3), irinotecan (30 mg/kg QD×6), and cisplatin (6 mg/kg Q4D×3) were used as the positive control and were administered as indicated. As shown in FIG. 2A, BO-1978 is more efficacious than etoposide, irinotecan and cisplatin. The doses used for each drug were within the maximum tolerated dose (MTD), which did not cause body weight lost (FIG. 2B). The mice in the control group, those treated with cisplatin, and those treated with etoposide were scarified on day 35, and those treated with irinotecan on day 45, because tumor size was over 2,500 mm³. One out of 4 mice treated with BO-1978 had a longer survival term (>430 days) as shown in FIG. 2C.

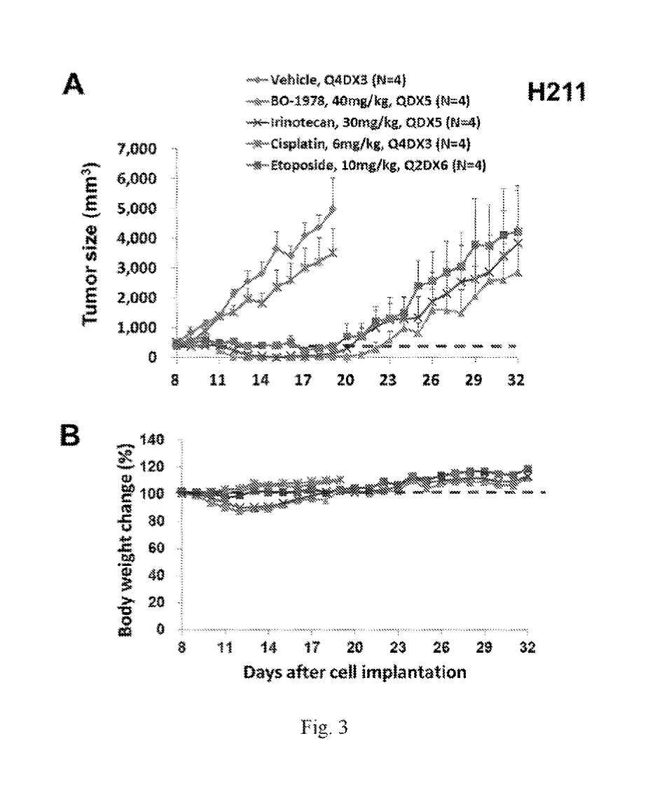

In another experiment, the effect of BO-1978 on the inhibition of SCLC H211 cells, a fast growing cell line, was evaluated in xenograft model. Mice were treated with the tested compounds under the same dosages and administration route as described above. As shown in FIG. 3A and B, BO-1978 is more potent than the drugs used for comparison. The present invention demonstrated that BO-1978 is superior to etoposide, irinotecan and cisplatin, which are widely used currently for the treatment of SCLC patients.

### Combination Therapy

### Example 3 Combination Treatment with BO-1978 and Irinotecan BO-1978 Synergistically Kills SCLC Cells

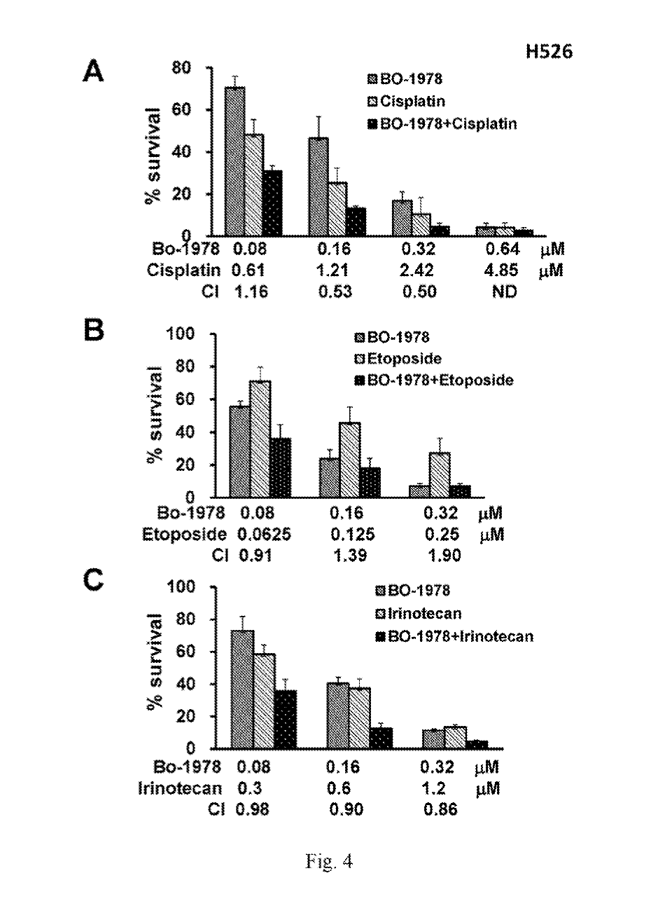

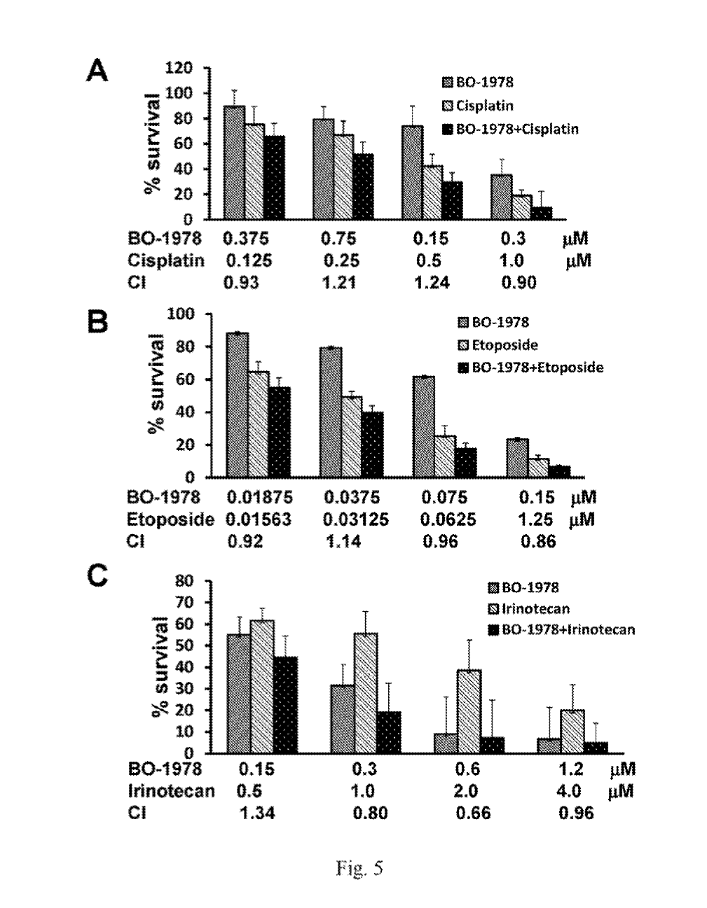

Combination therapy is therapy that uses more than one drug for therapy. It is well known that chemotherapy drugs are most effective when they are administered with other drugs that have different mechanism of action, thereby decreasing the possibility of drug resistance developing in cancer cells. To examine whether BO-1978 is useful in combination therapy, an alamarBlue assay was performed to demonstrate whether there is enhanced cytotoxicity by co-treatment with BO-1978 and other therapeutic agents, such as cisplatin, etoposide, and irinotecan in H526 and H211 cells in the toxic dose range. Notably, it was found that BO-1978 enhanced the cytotoxicity in both H526 and H211 cells when co-treated with irinotecan at various ratios as shown in FIGS. 4 and 5. The resulting combination index (CI) theorem of Chou-Talalay offers quantitative definition for additive effect (CI=1), synergism (CI<1), and antagonism (CI>1) in drug combinations (Chou 2010⁴⁴).

### Example 4 Effective Suppression of SCLC Cells by Combination Treatment with BO-1978 and Irinotecan in Xenograft Model

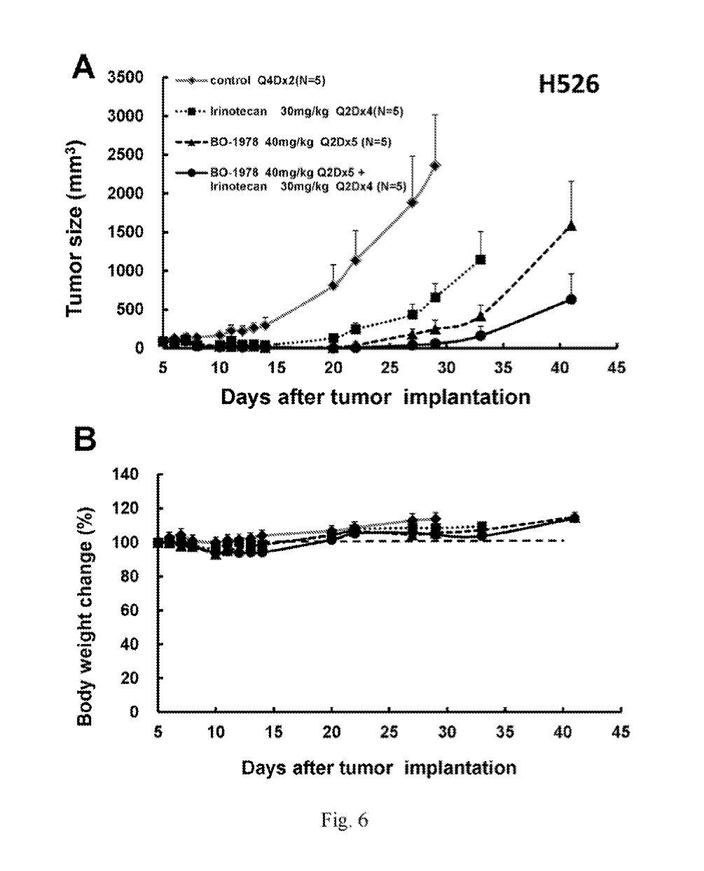

In addition to enhancement by BO-1978 of the cytotoxicity in H526 and H211 cells when co-treated with irinotecan, the therapeutic efficacy of BO-1978 or irinotecan (alone) and BO-1978 in combination with irinotecan was evaluated in nude mice bearing SCLC H526 xenograft model. The mice (n=5) bearing SCLC H526 xenografts were treated with BO-1978 (alone, 40 mg/kg, Q2D×5), irinotecan (alone, 30 mg/kg, Q2D×5), and BO-1978 (40 mg/kg)+irinotecan (30 mg/kg) (Q2D×5) via iv inj. As shown in FIG. 6, BO-1978 (alone) significantly suppressed tumor growth, 1/5<100 mm3 and 4/5 complete remission (CR) on day 14 (D14). Remarkably, tumor CR was observed in the mice treated with BO-1978+irinotecan, 5/3 CR on D20, 1/5<100 mm³ and 4/5 CR on D22, and 1/5 CR on D149. However, the tumor relapsed on day 15 in the mice after treatment with irinotecan alone.

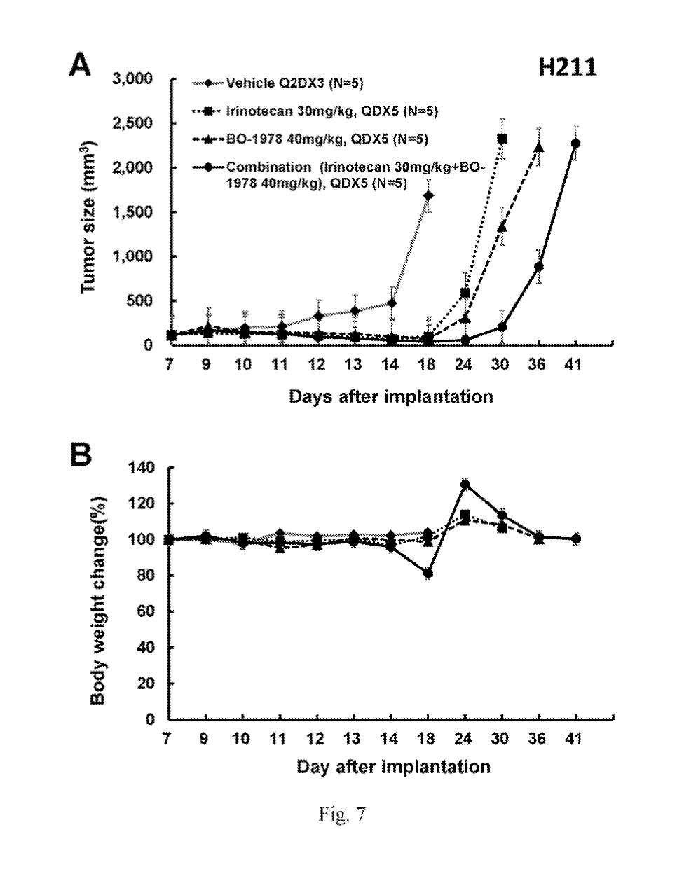

In another experiment, the therapeutic efficacy of BO-1978 (alone) or in combination with irinotecan against SCLC H211 was investigated in xenograft models (FIG. 7). The H211 bearing mice (n=5) were administered with BO-1978 (alone, 40 mg/kg, QD×5), irinotecan (alone, 30 mg/kg, QD×5), and BO-1978 (40 mg/kg)+irinotecan (30 mg/kg) (QD×5) via iv inj. In the mono-drug treated mice, BO-1978 (alone) was more potent than irinotecan (alone); tumor CR was seen on day 14 when mice were treated with BO1978 (alone), but showed tumor relapse on day 18. Notably, tumor CR was observed on day 18 in the mice treated with BO-1978+irinotecan; however, tumor relapsed on day 24.

### Combination Therapy of BO-1978 with Targeted Therapeutics Against DNA, Damage Response (DDR) in SCLC Cell Model

The molecules involved in DNA damage response (DDR) are potential targets for development of anticancer agents (O'Connor 2015⁴⁹). Dysregulation of DDR not only leads to mutagenesis and carcinogenesis but also cell death. Therapeutic agents targeting the principal DDR actors are demonstrated to trigger cell death and hence prevent cancer progression. However, whether the combination of DDR inhibitors with BO-1978 can be an effective strategy to increase the therapeutic efficacy of BO-1978 against a variety of cancers is still unknown.

### Example 5 Combination Therapy of BO-1978 and PARP Inhibitors (BMN-673 and HY-10130) Against SCLC

Poly(ADP-ribose) polymerase (PARP), a pivotal enzyme in the signaling to DNA repair, is activated by DNA strand breaks (Herriott et al. 2015⁵⁰). Inhibiting PARP catalysis blocks base excision repair (BER) and leads to the accumulation of unrepaired DNA single-strand breaks (SSBs), which are subsequently converted into double-strand breaks (DSBs) in replicating cells. Such DSBs require competent homologous recombination (HR) repair to allow cell survival. Unfortunately, HR repair is defective in some cancer patients. Currently, PARP inhibitors are pursued as a drug target in several clinical trials (Benafif and Hall 2015⁵¹).

Talazoparib (BMN 673: (8S,9R)-5-Fluoro-8-(4-fluorophenyl)-9-(1-methyl-1H-1,2,4-triazol-5-yl)-2,7,8,9-tetrahydro-3H-pyrido[4,3,2-de]phthalazin-3-one), and oral PARP1/2 inhibitor, has been shown to display single-agent synthetic lethality in BRCA1/2 and PTEN-deficient cell lines but also potently inhibit the growth of tumors harboring mutations in DNA repair pathways in animal models (Minami et al. 2013⁵²; Shen et al. 2013⁵³; Andrei et al. 2015⁵⁴). It is especially shows promise as single-agent treatment in patients with non-small cell lung cancer (NSCLC), small cell lung cancer (SCLC), advanced ovarian and breast cancer harboring deleterious BRCA1/2 mutations. However, BMN 673 has demonstrated enhancement of antitumor effects of TMZ, cisplatin (CIS), and carboplatin (Engert et al. 2015⁵⁵; Engert et al. 2017⁵⁶). Veliparib (HY-10130), also an orally active PARP inhibitor (Donawho et al. 2007⁵⁷), has been shown to facilitate the therapeutic effects of fractionated radiation through its impairment of single- and double-strand break repair pathways (Barazzuol et al. 2013⁵⁸). Besides ionizing radiation, HY-10130 (Veliparib dihydrochloride, 2-((R)-2-Methylpyrrolidin-2-yl)-1H-benzimidazole-4-carboxamide) potentiates the anticancer activity of temozolomide, cisplatin, carboplatin, and cyclophosphamide in a variety of tumors (Hussain et al. 2014⁵⁹; Wagner 2015⁶⁰).

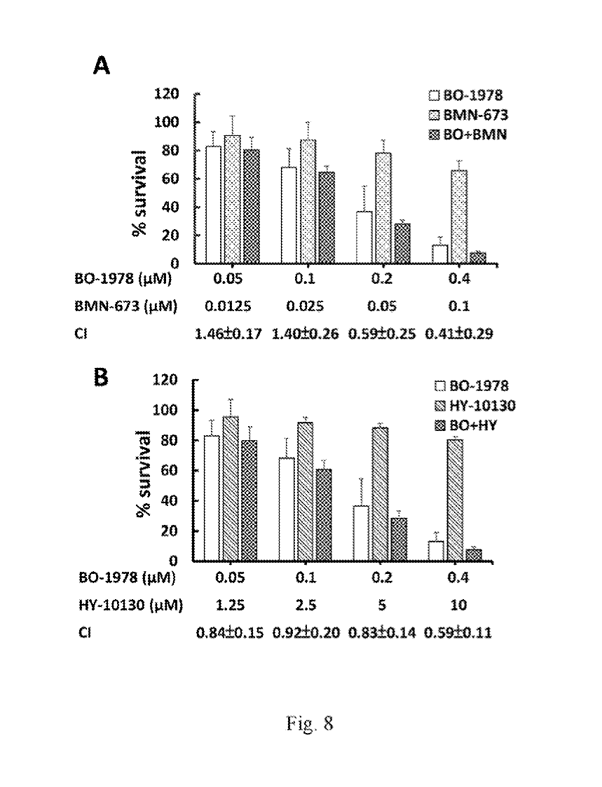

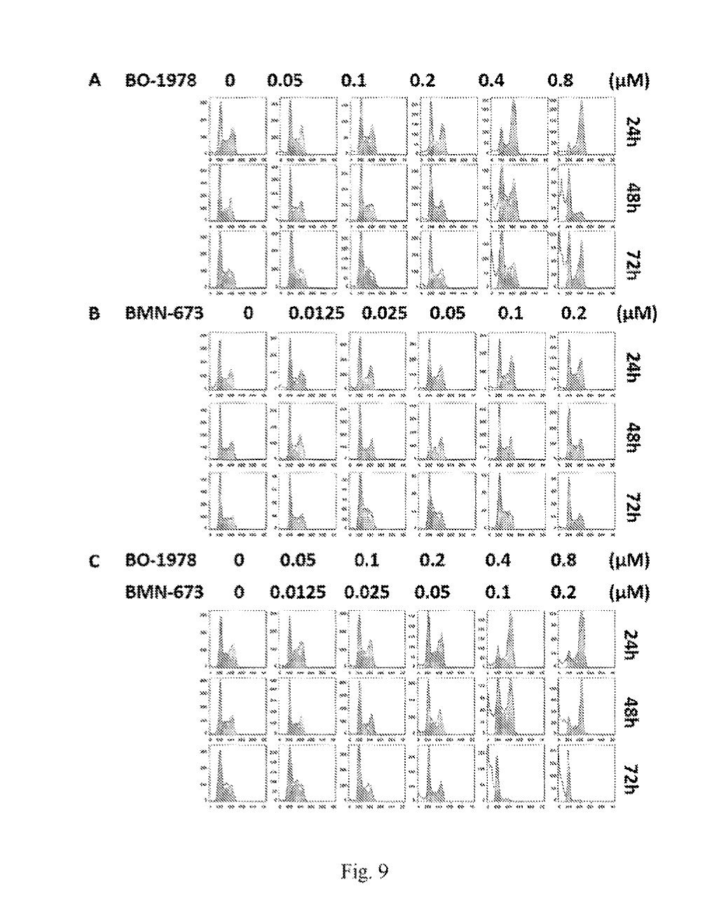

In the present invention, the SCLC H211 cells were treated with the combination of BO-1978 and BMN 673 or HY-10130 for 72 h. Cell survival was analyzed by PrestoBlue Assay. The combination index (CI) was calculated using CompuSyn software (Chou 2006⁶¹). When CI values are <1, the combination displays synergistic suppression of cell growth. As shown in FIGS. 8A and 8B, combination of BO-1978 and BMN 673 or HY-10130 synergistically suppressed the growth of SCLC H211 cells. By aid of flow cytometric technique, BO-1978 induced severe G2/M arrest in H211 cells accompanied with the appearance of the subG1 cells at high doses (FIG. 9A), whereas BMN 673 delayed the progression of the G2/M phase but did not induce subG1 cells (FIG. 9B). In the combination of BO-1078 and BMN 673, similar G2/M arrest was observed as BO-1978 (FIG. 9C). However, most cells treated with high doses of BO-1978+BMN 673 appealed at the subG1 phase at 72 h. Since the subG1 cells indicate apoptotic death, these results indicate that the combination of BO-1978+BMN 673 may mediate through unrepaired DNA to synergistically trigger the execution of apoptosis.

### Example 6 Combination Therapy of BO-1978 and PI3K Inhibitor (LY294002)

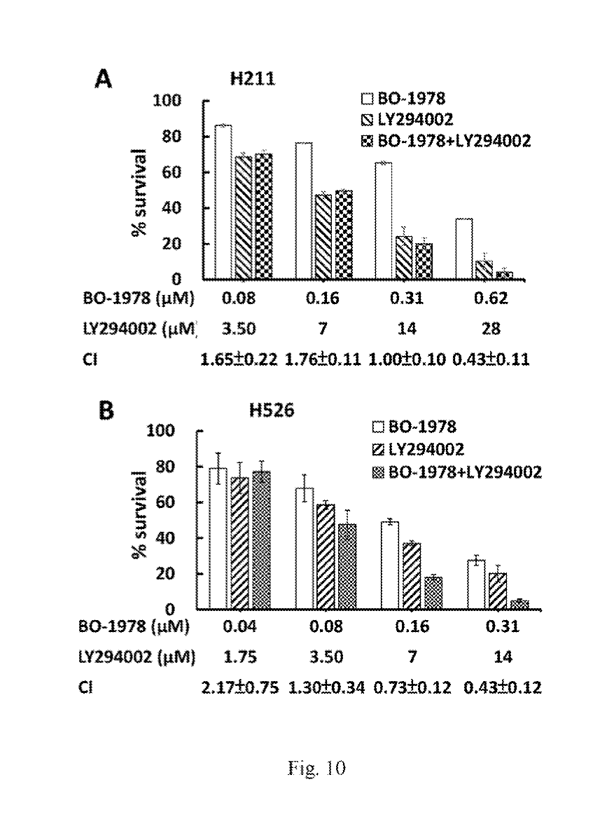

The PI3K/AKT pathway controls a number of cellular processes including cytoskeletal organization, cell growth and survival (Engelman et al. 2006⁶²). In response to DNA damage, PI3K/AKT signaling activates homologous recombination (HR) and non-homologous end joining (NHEJ) pathways to repair the damaged DNA (Deng et al. 2011⁶³). Many specific inhibitors of various PI3K isoforms have been used in clinical trials (Garcia-Echeverria and Sellers 2008⁶⁴; Liu et al. 2009⁶⁵). LY294002 (2-Morpholin-4-yl-8-phenylchromen-4-one), the first synthetic inhibitor without selectivity for individual isoforms of PI3K and ATM (Garcia-Echeverria and Sellers 2008⁶⁴; Liu et al. 2009⁶⁵), has been used in combination with chemotherapeutic agents and ionizing radiation (Hu et al. 2002⁶⁶; Lee et al. 2006⁶⁷). The clinical use of LY294002 is limited because of its toxicity and low solubility. However, it has been used extensively in various in vitro and in vivo systems to evaluate the biologic significance of PI3K (Liu et al. 2009⁶⁵). The experiments of the present invention show the synergistic cytotoxic effects of BO-1978 and LY294002 in two SCLC cell lines, H211 and H526. As shown in FIG. 10, at certain combination doses, the CL values were <1, indicating the synergistic effects of BO-1978 and LY294002 on suppression the growth of SCLC cells.

In summary, the experiments indicate that the combination of BO-1978 with a DDR inhibitor may synergistically enhance their therapeutic efficacies against SCLC.

### REFERENCES

1. Parkin D M, Bray F, Ferlay J, Pisani P (2005) Global cancer statistics, 2002. CA Cancer J Clin 55:74-108

2. Edwards B K, Noone A M, Mariotto A B, et al. (2014) Annual Report to the Nation on the status of cancer, 1975-2010, featuring prevalence of comorbidity and impact on survival among persons with lung, colorectal, breast, or prostate cancer. Cancer 120:1290-1314

3. Biesalski H K, Bueno de Mesquita B, Chesson A, et al. (1998) European Consensus Statement on Lung Cancer: risk factors and prevention. Lung Cancer Panel. CA Cancer J Clin 48:167-176; discussion 164-166

4. Lynch T J, Bell D W, Sordella R, et al. (2004) Activating mutations in the epidermal growth factor receptor underlying responsiveness of non-small-cell lung cancer to gefitinib. The New England journal of medicine 350:2129-2139

5. Sasaki T, Rodig S J, Chirieac L R, Janne P A (2010) The biology and treatment of EML4-ALK non-small cell lung cancer. Eur J Cancer 46:1773-1780

6. Clegg A, Scott D A, Hewitson P, Sidhu M, Waugh N (2002) Clinical and cost effectiveness of paclitaxel, docetaxel, gemcitabine, and vinorelbine in non-small cell lung cancer: a systematic review. Thorax 57:20-28

7. Cataldo V D, Gibbons D L, Perez-Soler R, Quintas-Cardama A (2011) Treatment of non-small-cell lung cancer with erlotinib or gefitinib. The New England journal of medicine 364:947-955

8. Chang A (2011) Chemotherapy, chemoresistance and the changing treatment landscape for NSCLC. Lung cancer 71:3-10

9. Karachaliou N, Pilotto S, Lazzari C, Bria E, de Marinis F, Rosell R (2016) Cellular and molecular biology of small cell lung cancer: an overview. Transl Lung Cancer Res 5:2-15

10. Pleasance E D, Stephens P J, O'Meara S, et al. (2010) A small-cell lung cancer genome with complex signatures of tobacco exposure. Nature 463:184-190

11. WHO (1981) Histological Typing of Lung Tumors. 2 edn. World Health Organization, Geneva

12. Murray N, Coy P, Pater J L, et al. (1993) Importance of timing for thoracic irradiation in the combined modality treatment of limited-stage small-cell lung cancer. The National Cancer Institute of Canada Clinical Trials Group. Journal of clinical oncology: official journal of the American Society of Clinical Oncology 11:336-344

13. Shepherd F A, Crowley J, Van Houtte P, et al. (2007) The International Association for the Study of Lung Cancer lung cancer staging project, proposals regarding the clinical staging of small cell lung cancer in the forthcoming (seventh) edition of the tumor, node, metastasis classification for lung cancer. Journal of thoracic oncology: official publication of the International Association for the Study of Lung Cancer 2:1067-1077

14. Kalemkerian G P (2014) Advances in pharmacotherapy of small cell lung cancer. Expert Opin Pharmacother 15:2385-2396

15. Fukuoka M, Furuse K, Saijo N, et al. (1991) Randomized trial of cyclophosphamide, doxorubicin, and vincristine versus cisplatin and etoposide versus alternation of these regimens in small-cell lung cancer. Journal of the National Cancer Institute 83:855-861

16. Alvarado-Luna G, Morales-Espinosa D (2016) Treatment for small cell lung cancer, where are we now?—a review. Transl Lung Cancer Res 5:26-38

17. Byers L A, Rudin C M (2015) Small cell lung cancer: where do we go from here? Cancer 121:664-672

18. Murray N, Turrisi A T, 3rd (2006) A renew of first-line treatment for small-cell lung cancer. Journal of thoracic oncology: official publication of the International Association for the Study of Lung Canter 1:270-278

19 Noda K, Nishiwaki Y, Kawahara M, et. al. (2002) Irinotecan plus cisplatin compared with etoposide plus cisplatin for extensive small-cell lung cancer. The New England journal of medicine 346:85-91

20. Sundstrom S, Bremnes R M, Kaasa S, et al. (2002) Cisplatin and etoposide regimen is superior to cyclophosphamide, epirubicin, and vincristine regimen in small-cell lung cancer: results from a randomized phase III trial with 5 years' follow-up. Journal of clinical oncology: official journal of the American Society of Clinical Oncology 20:4665-4672

21. Eckardt J R, von Pawel J, Papai Z, et al. (2006) Open-label, multicenter, randomized, phase III study comparing oral topotecan/cisplatin versus etoposide/cisplatin as treatment for chemotherapy-naive patients with extensive-disease small-cell lung cancer. Journal of clinical oncology: official journal of the American Society of Clinical Oncology 24:2044-2051

22. Hanna N, Bunn P A, Jr., Langer C, et al. (2006) Randomized III trial comparing irinotecan/cisplatin with etoposide/cisplatin in patients with previously untreated extensive-stage disease small-cell lung cancer. Journal of clinical oncology: official journal of the American Society of Clinical Oncology 24:2038-2043

23. Heigener D F, Manegold C, Jager E, Saal J G, Zuna I, Gatzemeier U (2009) Multicenter randomized open-label phase III study comparing efficacy, safely, and tolerability of conventional carboplatin plus etoposide versus dose-intensified carboplatin plus etoposide plus lenograstim in small-cell lung cancer in “extensive disease” stage. Am J Clin Oncol 32:61-64

24. Lara P N, Jr., Natale R, Crowley J, et al. (2009) Phase III trial of irinotecan/cisplatin compared with etoposide/cisplatin in extensive-stage small-cell lung cancer, clinical and pharmacogenomic results from SWOG S0124. Journal of clinical oncology: official journal of the American Society of Clinical Oncology 27:2530-2535

25. Brahmer J R, Ettinger D S (1998) Carboplatin in the Treatment of Small Cell Lung Cancer. Oncologist 3:143-154

26. Azim H A, Jr., Ganti A K (2007) Treatment options for relapsed small-cell lung cancer. Anti-cancer drugs 18:255-261

27. Horn L, Dahlberg S E, Sandler A B, et al. (2009) Phase II study of cisplatin plus etoposide and bevacizumab for previously untreated, extensive-stage small-cell lung cancer. Eastern Cooperative Oncology Group Study E3501. Journal of clinical oncology: official journal of the American Society of Clinical Oncology 27:6006-6011

28. Luo J, Wu F Y, Li A W, Zheng D, Liu J M (2012) Comparison of vinorelbine, ifosfamide and cisplatin (NIP) and etoposide and cisplatin (EP) for treatment of advanced combined small cell lung cancer (cSCLC) patients: a retrospective study. Asian Pac J Cancer Prev 13:4703-4706

29. Slotman B J, van Tinteren H, Praag J O, et al. (2015) Use of thoracic radiotherapy for extensive stage small-cell lung cancer: a phase 3 randomised controlled trial. Lancet 385:36-42

30. Livingston R B, Moore T N, Heilbrun L, et al. (1978) Small-cell carcinoma of the lung: combined chemotherapy and radiation: a Southwest Oncology Group study. Ann Intern Med 88:194-199

31. Feld R, Pringle J F, Evans W K, et al. (1981) Combined modality treatment of small cell carcinoma of the lung. Arch Intern Med 141:469-473

32. Santarpia M, Daffina M G, Karachaliou N, et al. (2016) Targeted drugs in small-cell lung cancer. Transl Lung Cancer Res 5:51-70

33. Abidin A Z, Garassino M C, Califano R, Harle A, Blackhall F (2010) Targeted therapies in small cell lung cancer: a review. Ther Adv Med Oncol 2:25-37

34. Koinis F, Kotsakis A, Georgoulias V (2016) Small cell lung cancer (SCLC): no treatment advances in recent years. Transl Lung Cancer Res 5:39-50

35. Guan H, Chen H, Peng W, et al. (2006) Design of beta-carboline derivatives as DNA-targeting antitumor agents. Eur J Med Chem 41:1167-1179

36. Funayama Y, Nishio K, Wakabayashi K, et al. (1996) Effects of beta- and gamma-carboline derivatives of DNA topoisomerase activities. Mutation research 349:183-191

37. Deveau A M, Labroli M A, Dieckhaus C M, Barthen M T, Smith K S, Macdonald T L (2001) The synthesis of amino-acid functionalized beta-carbolines as topoisomerase II inhibitors. Bioorganic & medicinal chemistry letters 11:1251-1255

38. Song Y, Kesuma D, Wang J, et al. (2004) Specific inhibition of cyclin-dependent kinases and cell proliferation by harmine. Biochem Biophys Res Commun 317:128-132

39. An Castro A C, Dang L C, Soucy F, et al. (2003) Novel IKK inhibitors: beta-carbolines. Bioorganic & medicinal chemistry letters 13:2419-2422

40. Anderson W K, Halat M J (1979) Antileukemic activity of derivatives of 1,2-dimethyl-3,4-bis(hydroxymethyl)-5-phenylpyrrole bis(N-methylcarbamate). Journal of medicinal chemistry 22:977-980

41. Anderson W K, New J S, Corey P F (1980) Vinylogous carbinolamine tumor inhibitors 7. Tumor inhibitory agents: bis-(N-alkylcarbamate) derivatives of 2,3-dihydro-5-(3′,4′-dichlorophenyl)-6,7-bis(hydroxymethyl-1H-pyrrolizine. Arzneimittelforschung 30:765-767

42. Chen C W, Wu M H, Chen Y F, et al. (2016) A Potent Derivative of Indolizino[6,5-b]Indole for Treatment of Human Non-Small Cell Lung Cancer Cells. Neoplasia 18:199-212

43. Zarogoulidis K, Zarogoulidis P, Darwiche K, et al. (2013) Treatment of non-small cell lung cancer (NSCLC). Journal of thoracic disease 5 Suppl 4:S389-396

44. Chou T C (2010) Drug combination studies and their synergy quantification using the Chou-Talalay method. Cancer research 70:440-446

45. Govindan R, Page N, Morgensztern D, et al. (2006) Changing epidemiology of small-cell lung cancer in the United States over the last 30 years: analysis of the surveillance, epidemiologic, and end results database. Journal of clinical oncology: official journal of the American Society of Clinical Oncology 24:4539-4544

46. Jemal A, Murray T, Ward E, et al. (2005) Cancer statistics, 2005. CA: a cancer journal for clinicians 55:10-30

47. Prasad U S, Naylor A R, Walker W S, Lamb D, Cameron E W, Walbaum P R (1989) Long term survival after pulmonary resection for small cell carcinoma of the lung. Thorax 44:784-787

48. Chaniyara R, Tala S, Chen C W, et al. (2013) Novel antitumor indolizino[6,7-b]indoles with multiple modes of action: DNA cross-linking and topoisomerase I and II inhibition. Journal of medicinal chemistry 56:1544-1563

49. O'Connor M J (2015) Targeting the DNA Damage Response in Cancer. Mol Cell 60:547-560

50. Herriott A, Tudhope S J, Junge G, et al. (2015) PARP1 expression, activity and ex vivo sensitivity to the PARP inhibitor, talazoparib (BMN 673), in chronic lymphocytic leukaemia. Oncotarget 6:43978-43991

51. Benafif S, Hall M (2015) An update on PARP inhibitors for the treatment of cancer. Onco Targets Ther 8:519-528

52. Minami D, Takigawa N, Takeda H, et al. (2013) Synergistic effect of olaparib with combination of cisplatin on PTEN-deficient lung cancer cells. Molecular cancer research: MCR 11:140-148

53. Shen Y, Rehman F L, Feng Y, et al. (2013) BMN 673, a novel and highly potent PARP1/2 inhibitor for the treatment of human cancers with DNA repair deficiency. Clinical cancer research: an official journal of the American Association for Cancer Research 19:5003-5015

54. Andrei A Z, Hall A, Smith A L, et al. (2015) Increased in vitro and in vivo sensitivity of BRCA2-associated pancreatic cancer to the poly(ADP-ribose) polymerase-1/2inhibitor BMN 673. Cancer Lett 364:8-16

55. Engert F, Schneider C, Weibeta L M, Probst M, Fulda S (2015) PARP Inhibitors Sensitize Ewing Sarcoma Cells to Temozolomide-Induced Apoptosis via the Mitochondrial Pathway. Molecular cancer therapeutics 14:2818-2830

56. Engert F, Kovac M, Baumhoer D, Nathrath M, Fulda S (2017) Osteosarcoma cells with genetic signatures of BRCAness are susceptible to the PARP inhibitor talazoparib alone or in combination with chemotherapeutics. Oncotarget 8:48794-48806

57. Donawho C K, Luo Y, Luo Y, et al. (2007) ABT-888, an orally active poly(ADP-ribose) polymerase inhibitor that potentiates DNA-damaging agents in preclinical tumor models. Clinical cancer research: an official journal of the American Association for Cancer Research 13:2728-2737

58. Barazzuol L, Jena R, Burnet N G, et al. (2013) Evaluation of poly (ADP-ribose) polymerase inhibitor ABT-888 combined with radiotherapy and temozolomide in glioblastoma. Radial Oncol 8:65

59. Hussain M, Carducci M A, Slovin S, et al. (2014) Targeting DNA repair with combination veliparib (ABT-888) and temozolomide in patients with metastatic castration-resistant prostate cancer. Invent New Drugs 32:904-912

60. Wagner L M (2015) Profile of veliparib and its potential in the treatment of solid tumors. Onco Targets Ther 8:1931-1939

61. Chou T C (2006) Theoretical basis, experimental design, and computerized simulation of synergism and antagonism in drug combination studies. Pharmacol Rev 58:621-681

62. Engelman J A, Luo J, Cantley L C (2006) The evolution of phosphatidylinositol 3-kinases as regulators of growth and metabolism. Nat Rev Genet 7:606-619

63. Deng R, Tang J, Ma J G, et al. (2011) PKB/Akt promotes DSB repair in cancer cells through upregulating Mre11 expression following ionizing radiation. Oncogene 30:944-955

64. Garcia-Echeverria C, Sellers W R (2008) Drug discovery approaches targeting the PI3K/Akt pathway in cancer. Oncogene 27:5511-5526

65. Liu P, Cheng H, Roberts T M, Zhao J J (2009) Targeting the phosphoinositide 3-kinase pathway in cancer. Nat Rev Drug Discov 8:627-644

66. Hu J, Hofmann J, Lu Y, Mills G B, Jaffe R B (2002) Inhibition of phosphatidylinositol 3′-kinase increases efficacy of paclitaxel in in vitro and in vivo ovarian cancer models. Cancer Res 62:1087-1092

67. Lee C M, Fuhrman C B, Planelles V, et al. (2006) Phosphatidylinositol 3-kinase inhibition by LY294002 radiosensitizes human cervical cancer cell lines. Clinical cancer research: an official journal of the American Association for Cancer Research 12:250-256

1. A method for treating small cell lung cancer (SCLC) in a subject in need thereof comprising administering to the subject a therapeutically effective amount of a compound of Formula I: 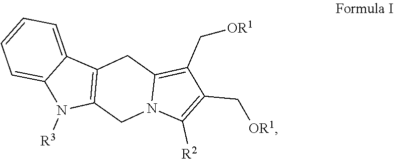
wherein:
R¹ is hydrogen or —C(═O)NHR; wherein R is unsubstituted or substituted alkyl group, unsubstituted or substituted alkenyl, unsubstituted or substituted alkynyl, unsubstituted or substituted cycloalkyl, unsubstituted or substituted aryl, unsubstituted or substituted heteroaryl, and unsubstituted or substituted benzyl;
R² is selected from the group consisting of hydrogen, unsubstituted or substituted alkyl, unsubstituted or substituted alkenyl, unsubstituted or substituted alkynyl, unsubstituted or substituted cycloalkyl, unsubstituted or substituted aryl, unsubstituted or substituted heteroaryl, and unsubstituted or substituted benzyl; and
R³ is selected from the group consisting of hydrogen, unsubstituted or substituted alkyl group, unsubstituted or substituted alkenyl, unsubstituted or substituted alkynyl, unsubstituted or substituted cycloalkyl, unsubstituted or substituted benzyl, an acyl (R^{a}CO), a methansulfonyl (Me₂SO₂) and a toluenesulfonyl (MeC₆H₄SO₂); wherein R^{a} is unsubstituted or substituted alkyl group, unsubstituted or substituted alkenyl group, unsubstituted or substituted alkynyl, unsubstituted or substituted cycloalkyl, unsubstituted or substituted aryl, unsubstituted or substituted heteroaryl, and unsubstituted or substituted benzyl,
or an enantiomer, diastereomer, racemate, pharmaceutically acceptable salt, solvate or prodrug thereof.

2. The method of claim 1, wherein:
(i) R¹ is hydrogen;
(ii) R² is ethyl; and/or;
(iii) R³ is methyl.

3. (canceled)

4. (canceled)

5. The method of claim 1, wherein R¹ is hydrogen, R² is ethyl and R³ is methyl.

6. The method of claim 1, wherein the compound of Formula I is selected from the group consisting of:
[3-Ethyl-6-methyl-6,11-dihydro-5H-indolizino[6,7-b]indole-1,2-diyl]dimethanol;
(3-(Phenyl)-6-methyl-6,11-dihydro-5H-indolizino[6,7-b]indole-1,2-diyl)dimethanol;
(3-(4-Fluorophenyl)-6-methyl-6,11-dihydro-5H-indolizino[6,7-b]indole-1,2-diyl)dimethanol;
(3-(4-Chlorophenyl)-6-methyl-6,11-dihydro-5H-indolizino[6,7-b]indole-1,2-diyl)dimethanol;
(3-(3,4-Difluorophenyl)-6-methyl-6,11-dihydro-5H-indolizino[6,7-b]indole-1,2-diyl)dimethanol;
[6-Methyl-3-phenyl-6,11-dihydro-5H-indolizino[6,7-b]indole-1,2-diyl]bis(methylene) bis(ethylcarbamate);
[3-(4-Fluorophenyl)-6-methyl-6,11-dihydro-5H-indolizino[6,7-b]indole-1,2-diyl]bis(methylene) bis(ethylcarbamate;
[3-(4-Chlorophenyl)-6-methyl-6,11-dihydro-5H-indolizino[6,7-b]indole-1,2-diyl]bis(methylene) bis(ethylcarbamate); and
[3-(4-Difluorophenyl)-6-methyl-6,11-dihydro-5H-indolizino[6,7-b]indole-1,2-diyl]bis(methylen) bis(ethylcarbamate).

7. The method of claim 1, wherein the compound of Formula I is [3-ethyl-6-methyl-6,11-dihydro-5H-indolizino[6,7-b]indole-1,2-diyl]dimethanol.

8. A method for treating small cell lung cancer (SCLC) in a subject in need thereof comprising administering to the subject a therapeutically effective amount of a compound of Formula I: 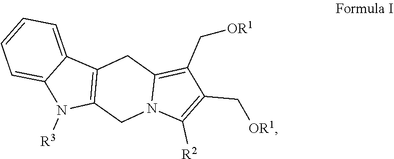
wherein
R¹ is hydrogen or —C(═O)NHR; wherein R is unsubstituted or substituted alkyl group, unsubstituted or substituted alkenyl, unsubstituted or substituted alkynyl, unsubstituted or substituted cycloalkyl, unsubstituted or substituted aryl, unsubstituted or substituted heteroaryl, and unsubstituted or substituted benzyl;
R² is selected from the group consisting of hydrogen, unsubstituted or substituted alkyl, unsubstituted or substituted alkenyl, unsubstituted or substituted alkynyl, unsubstituted or substituted cycloalkyl, unsubstituted or substituted aryl, unsubstituted or substituted heteroaryl, and unsubstituted or substituted benzyl; and
R³ is selected from the group consisting of hydrogen, unsubstituted or substituted alkyl group, unsubstituted or substituted alkenyl, unsubstituted or substituted alkynyl, unsubstituted or substituted cycloalkyl, unsubstituted or substituted benzyl, an acyl (R^{a}CO), a methansulfonyl (Me₂SO₂) and a toluenesulfonyl (MeC₆H₄SO₂); wherein R^{a} is unsubstituted or substituted alkyl group, unsubstituted or substituted alkenyl group, unsubstituted or substituted alkynyl, unsubstituted or substituted cycloalkyl, unsubstituted or substituted aryl, unsubstituted or substituted heteroaryl, and unsubstituted or substituted benzyl,
or an enantiomer, diastereomer, racemate, pharmaceutically acceptable salt, solvate or prodrug thereof in combination with a second anticancer agent, a surgical therapy, a radiation therapy, a chemotherapy, a targeted therapy, or combination thereof.

9. The method of claim 8, wherein:
(i) R¹ is hydrogen;
(ii) R² is ethyl; and/or
(iii) R³ is methyl.

10. (canceled)

11. (canceled)

12. The method of claim 8, wherein R¹ is hydrogen, R² is ethyl and R³ is methyl.

13. The method of claim 8, wherein the compound of Formula I is selected from the group consisting of:
[3-Ethyl-6-methyl-6,11-dihydro-5H-indolizino[6,7-b]indole-1,2-diyl]dimethanol;
(3-(Phenyl)-6-methyl-6,11-dihydro-5H-indolizino[6,7-b]indole-1,2-diyl)dimethanol;
(3-(4-Fluorophenyl)-6-methyl-6,11-dihydro-5H-indolizino[6,7-b]indole-1,2-diyl)dimethanol;
(3-(4-Chlorophenyl)-6-methyl-6,11-dihydro-5H-indolizino[6,7-b]indole-1,2-diyl)dimethanol;
(3-(3,4-Difluorophenyl)-6-methyl-6,11-dihydro-5H-indolizino[6,7-b]indole-1,2-diyl)dimethanol;
[6-Methyl-3-phenyl-6,11-dihydro-5H-indolizino[6,7-b]indole-1,2-diyl]bis(methylene) bis(ethylcarbamate);
[3-(4-Fluorophenyl)-6-methyl-6,11-dihydro-5H-indolizino[6,7-b]indole-1,2-diyl]bis(methylene) bis(ethylcarbamate;
[3-(4-Chlorophenyl)-6-methyl-6,11-dihydro-5H-indolizino[6,7-b]indole-1,2-diyl]bis(methylene) bis(ethylcarbamate); and
[3-(4-Difluorophenyl)-6-methyl-6,11-dihydro-5H-indolizino[6,7-b]indole-1,2-diyl]bis(methylen) bis(ethylcarbamate).

14. The method of claim 8, wherein the second anticancer agent is selected from the group consisting of anti-microtubule agents (such as diterpenoids and vinca alkaloids like vinorelbine); platinum coordination complexes; alkylating agents (such as nitrogen mustards like ifosphamide, oxazaphosphorines, alkylsulfonates, nitrosoureas (including 2-chloroethyl-3-sarcosinamide-1-nitrosourea (SarCNU)), busulfan, chloroambucil, cyclophosphamide, iphosphamide, melphalan, streptozocin, thiotepa, uracil nitrogen mustard, triethylenemelamine, temozolomide, and triazenes); antibiotic agents or plant alkaloids (such as cryptophycins, daunorubicin, doxorubicin, idarubicin, irinotecan, L-asparaginase, mitomycin-C, mitramycin, navelbine, paclitaxel, docetaxel, topotecan, vinblastine, vincristine, teniposide (VM-26), and etoposide (VP-16), anthracyclins, actinomycins (such as actinomycin-D) and bleomycins); topoisomerase II inhibitors (such as epipodophyllotoxins); hormones or steroids (such as 5α-reductase inhibitor, aminoglutethimide, anastrozole, bicalutamide, chlorotrianisene, diethylstilbestrol (DES), dromostanolone, estramustine, ethinyl estradiol, flutamide, fluoxymesterone, goserelin, hydroxyprogesterone, letrozole, leuprolide, medroxyprogesterone acetate, megestrol acetate, methyl prednisolone, methyltestosterone, mitotane, nilutamide, prednisolone, arzoxifene (SERM-3), tamoxifen, testolactone, testosterone, triamicnolone, and zoladex); synthetics (such as all-trans retinoic acid, carmustine (BCNU), carboplatin (CBDCA), lomustine (CCNU), cis-diaminedichloroplatinum (cisplatin), dacarbazine, gliadel, hexamethylmelamine, hydroxyurea, levamisole, mitoxantrone, o,p′-dichlorodiphenyldichloroethane (o,p′-DDD) (also known as lysodren or mitotane), oxaliplatin, porfimer sodium, procarbazine, and imatinib mesylate (Gleevec®)); antimetabolites (such as chlorodeoxyadenosine, cytosine arabinoside, 2′-deoxycoformycin, fludarabine phosphate, 5-fluorouracil (5-FU), 5-fluoro-2′-deoxyuridine (5-FUdR), gemcitabine, camptothecin, 6-mercaptopurine, methotrexate, 4-methylthioamphetamine (4-MTA), thioguanine, pemetrexed, purine and pyrimidine analogues and anti-folate compounds); biologies (such as alpha interferon, BCG (Bacillus Calmette-Guerin), granulocyte colony stimulating factor (G-CSF), granulocyte-macrophage colony-stimulating factor (GM-CSF), interleukin-2, and herceptin); topoisomerase I inhibitors (such as camptothecins; hormones and hormonal analogues); signal transduction pathway inhibitors (such as tyrosine receptor inhibitors like erlotinib; EGFR inhibitors like gefitinib and afatinib; TNFR inhibitors like denosumab); non-receptor tyrosine kinase angiogenesis inhibitors; immunotherapeutic agents; proapoptotic agents; epigenetic or transcriptional modulators (such as histone deacetylase inhibitors); DNA replication or transcription inhibitors (such as picoplatin); DNA damage response (DDR) inhibitors (such as Poly(ADP-ribose) polymerase (PARP) inhibitors (e.g., Talazoparib ((8S,9R)-5-Fluoro-8-(4-fluorophenyl)-9-(1-methyl-1H-1,2,4-triazol-5-yl)2,7,8,9-tetrahydro-3H-pyrido[4,3,2-de]phthalazin-3-one), Veliparib (HY-10130; 2-((R)-2-Methylpyrrolidin-2-yl)-1H-benzimidazole-4-carboxamide), Olaparib (4-[[3-[4-(cyclopropanecarbonyl)piperazine-1-carbonyl]-4-fluorophenyl]methyl]-2H-phthalazin-1-one, Niraparib 2-[4-[(3S)-piperidin-3-yl]phenyl]indazole-7-carboxamide), and Rucaparib (8-Fluoro-2-{4-[(methylamino)methyl]phenyl}-1,3,4,5-tetrahydro-6H-azepino[5,4,3-cd]indol-6-one)) or PI3K/AKT pathway inhibitors (e.g., LY294002 2-Morpholin-4-yl-8-phenylchromen-4-one), buparlisib (5-[2,6-bis(morpholin-4-yl)pyrimidin-4-yl]-4-(trifluoromethyl)pyridin-2-amine), and alpelisib ((2S)-1-N-[4-methyl-5-[2-(1,1,1-trifluoro-2-methylpropan-2-yl)pyridin-4-yl]1,3-thiazol-2-yl]pyrrolidine-1,2-dicarboxamide)); and cell cycle signaling inhibitors.

15. The method of claim 8, wherein the second anticancer agents is selected from the group consisting of irinotecan, etoposide, cisplatin, picoplatin, cyclophosphamide, doxorubicin, vincristine, topotecan, pemetrexed, carboplatin, gemcitabine, paclitaxel, vinorelbine, ifosphamide, erlotinib, gefitinib, afatinib, denosumab, Talazoparib, Veliparib, and LY294002.

16. The method of claim 8, wherein the compound of Formula I is [3-ethyl-6-methyl-6,11-dihydro-5H-indolizino[6,7-b]indole-1,2-diyl]dimethanol, and the second anticancer agent is irinotecan, etoposide, cisplatin, talazoparib, veliparib or LY294002.

17. A combination comprising the compound of Formula I 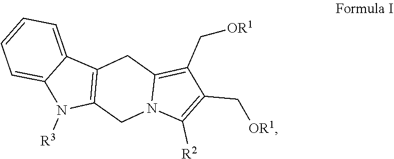
wherein
R¹ is hydrogen or —C(═O)NHR; wherein R¹ is unsubstituted or substituted alkyl group, unsubstituted or substituted alkenyl, unsubstituted or substituted alkynyl, unsubstituted or substituted cycloalkyl, unsubstituted or substituted aryl, unsubstituted or substituted heteroaryl, and unsubstituted or substituted benzyl;
R² is selected from the group consisting of hydrogen, unsubstituted or substituted alkyl, unsubstituted or substituted alkenyl, unsubstituted or substituted alkynyl, unsubstituted or substituted cycloalkyl, unsubstituted or substituted aryl, unsubstituted or substituted heteroaryl, and unsubstituted or substituted benzyl; and
R³ is selected from the group consisting of hydrogen, unsubstituted or substituted alkyl group, unsubstituted or substituted alkenyl, unsubstituted or substituted alkynyl, unsubstituted or substituted cycloalkyl, unsubstituted or substituted benzyl, an acyl (R^{a}CO), a methansulfonyl (Me₂SO₂) and a toluenesulfonyl (MeC₆H₄SO₂); wherein R^{a} is unsubstituted or substituted alkyl group, unsubstituted or substituted alkenyl group, unsubstituted or substituted alkynyl, unsubstituted or substituted cycloalkyl, unsubstituted or substituted aryl, unsubstituted or substituted heteroaryl, and unsubstituted or substituted benzyl,
or an enantiomer, diastereomer, racemate, pharmaceutically acceptable salt, solvate or prodrug thereof,
and a second anticancer agent.

18. The combination of claim 17, wherein:
(i) R¹ is hydrogen;
(ii) R² is ethyl; and/or
(iii) R³ is methyl.

19. (canceled)

20. (canceled)

21. The combination of claim 17, wherein R¹ is hydrogen, R² is ethyl and R³ is methyl.

22. The combination of claim 17, wherein the compound of Formula I is selected from the group consisting of:
[3-Ethyl-6-methyl-6,11-dihydro-5H-indolizino[6,7-b]indole-1,2-diyl]dimethanol;
(3-Phenyl)-6-methyl-6,11-dihydro-5H-indolizino[7,6-b]indole-1,2-diyl)dimethanol;
(3-(4-Fluorophenyl)-6-methyl-6,11-dihydro-5H-indolizino[6,7-b]indole-1,2-diyl)dimethanol;
(3-(4-Chlorophenyl)-6-methyl-6,11-dihydro-5H-indolizino[6,7-b]indole-1,2-diyl)dimethanol;
(3-(3,4-Difluorophenyl)-6-methyl-6,11-dihydro-5H-indolizino[6,7-b]indole-1,2-diyl)dimethanol;
[6-Methyl-3-phenyl-6,11-dihydro-5H-indolizino[6,7-b]indole-1,2-diyl]bis(methylene) bis(ethylcarbamate);
[3-(4-Fluorophenyl)-6-methyl-6,11-dihydro-5H-indolizino[6,7-b]indole-1,2-diyl]bis(methylene) bis(ethylcarbamate;
[3-(4-Chlorophenyl)-6-methyl-6,11-dihydro-5H-indolizino[6,7-b]indole-1,2-diyl]bis(methylene) bis(ethylcarbamate); and
[3-(4-Difluorophenyl)-6-methyl-6,11-dihydro-5H-indolizino[6,7-b]indole-1,2-diyl]bis(methylen) bis(ethylcarbamate).

23. The combination of claim 17, wherein the second anticancer agent is selected from the group consisting of anti-microtubule agents (such as diterpenoids and vinca alkaloids like vinorelbine); platinum coordination complexes; alkylating agents (such as nitrogen mustards like ifosphamide, oxazaphosphorines, alkylsulfonates, nitrosoureas (including 2-chloroethyl-3-sarcosinamide-1-nitrosourea (SarCNU)), busulfan, chlorambucil, cyclophosphamide, iphosphamide, melphalan, streptozocin, thiotepa, uracil nitrogen mustard, triethylenemelamine, temozolomide, and triazenes); antibiotic agents or plant alkaloids (such as cryptophycins, daunorubicin, doxorubicin, idarubicin, irinotecan, L-asparaginase, mitomycin-C, mitramycin, navelbine, . paclitaxel, docetaxel, topotecan, vinblastine, vincristine, teniposide (VM-26), and etoposide (VP-16), anthracyclins, actinomycins (such as actinomycin-D) and bleomycins); topoisomerase II inhibitors (such as epipodophyllotoxins); hormones or steroids (such as 5α-reductase inhibitor, aminoglutethimide, anastrozole, bicalutamide, chlorotrianisene, diethylstilbestrol (DES), dromostanolone, estramustine, ethinyl estradiol, flutamide, fluoxymesterone, goserelin, hydroxyprogesterone, letrozole, leuprolide, medroxyprogesterone acetate, megestrol acetate, methyl prednisolone, methyltestosterone, mitotane, nilutamide. prednisolone, arzoxifene (SERM-3), tamoxifen, testolactone, testosterone, triamicnolone, and zoladex); synthetics (such as all-trans retinoic acid, carmustine (BCNU), carboplatin (CBDCA), lomustine (CCNU), cis-diaminedichloroplatinum (cisplatin), dacarbazine, gliadel, hexamethylmelamine, hydroxyurea, levamisole, mitoxantrone, o,p′-dichlorodiphenyldichloroethane (o,p′-DDD) (also known as lysodren or mitotane), oxaliplatin, porfimer sodium, procarbazine, and imatinib mesylate (Gleevec®)); antimetabolites (such as chlorodeoxyadenosine, cytosine arabinoside, 2′-deoxycoformycin, fludarabine phosphate, 5-fluorouracil (5-FU), 5-fluoro-2′-deoxyuridine (5-FUdR), gemcitabine, camptothecin, 6-mercaptopurine, methotrexate, 4-methylthioamphetamine (4-MTA), thioguanine, pemetrexed, purine and pyrimidine analogues and anti-folate compounds); biologies (such as alpha interferon, BCG (Bacillus Calmette-Guerin), granulocyte colony stimulating factor (G-CSF), granulocyte-macrophage colony-stimulating factor (GM-CSF), interleukin-2, and herceptin); topoisomerase I inhibitors (such as camptothecins; hormones and hormonal analogues); signal transduction pathway inhibitors (such as tyrosine receptor inhibitors like erlotinib; EGFR inhibitors like gefitinib and afatinib; TNFR inhibitors like denosumab); non-receptor tyrosine kinase angiogenesis inhibitors; immunotherapeutic agents; proapoptotic agents; epigenetic or transcriptional modulators (such as histone deacetylase inhibitors); DNA replication or transcription inhibitors (such as picoplatin); DNA damage response (DDR) inhibitors (such as Poly(ADP-ribose) polymerase (PARP) inhibitors (e.g., Talazoparib ((8S,9R)-5-Fluoro-8-(4- fluorophenyl)-9-(1-methyl-1H-1,2,4-triazol-5-yl)-2,7-8,9-tetrahydro-3H-pyrido[4,3,2-de]phthalazin-3-one, Veliparib (HY-10130; 2-((R)-2-Methylpyrrolidin-2-yl)-1H-benzimidazole-4-carboxamide), Olaparib (4-[[3-[4-(cyclopropanecarbonyl)piperazine-1-carbonyl]-4-fluorophenyl]methyl]-2H-phthalazin-1-one, Niraparib 2-[4-[(3S)-piperidin-3-yl]phenyl]indazole-7-carboxamide), and Rucaparib (8-Fluoro-2-{4-[(methylamino)methyl]phenyl}-1,3,4,5-tetrahydro-6H-azepino[5,4,3-cd]indol-6-one)) or PI3K/AKT pathway inhibitors (e.g., LY294002 (2-Morpholin-4-yl-8-phenylchromen-4-one), buparlisib (5-[2,6-bis(morpholin-4-yl)pyrimidin-4-yl]-4-(trifluoromethyl)pyridin-2-amine), and alpelisib ((2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide)); and cell cycle signaling inhibitors.

24. The combination of claim 17, wherein the second anticancer agents is selected from the group consisting of irinotecan, etoposide, cisplatin, picoplatin, cyclophosphamide, doxorubicin, vincristine, topotecan, pemetrexed, carboplatin, gemcitabine, paclitaxel, vinorelbine, ifosphamide, erlotinib, gefitinib, afatinib, denosumab, Talazoparib, Veliparib, and LY294002.

25. The combination of claim 17, wherein the compound of Formula I is [3-ethyl-6-methyl-6,11-dihydro-5H-indolizino[6,7-b]indole-1,2-diyl]dimethanol, and the second anticancer agent is irinotecan, etoposide, cisplatin, talazoparib, veliparib or LY294002.
